(19) United States
(12) Patent Application Publication
(10) Pub. No.: US 2022/0135968 A1
(43) Pub. Date: May 5, 2022

(54) TREATMENT OF SYNUCLEINOPATHY AND ANIMAL MODELS OF SYNUCLEINOPATHY

(71) Applicant: Jay DELA CRUZ, Moreno Valley, CA (US)

(72) Inventor: Jay DELA CRUZ, Moreno Valley, CA (US)

(73) Assignee: TORREY PINES LAW GROUP, PC, San Diego, CA (US)

(21) Appl. No.: 17/057,087

(22) PCT Filed: Jun. 14, 2019
(86) PCT No.: PCT/US19/37385

§ 371 Date: Jun. 9, 2021

Related U.S. Application Data
(60) Provisional application No. 62/685,868, filed on Jun. 15, 2018.
(60) Provisional application No. 62/685,985, filed on Jun. 16, 2018.

(51) Int. Cl. C12N 15/11; A61K 38/34; A61K 38/17; G01N 33/50; G01N 33/68

(52) U.S. Cl. CPC C12N 15/111; A61K 38/34; A61K 38/1729; C12N 2310/20; G01N 33/6893; G01N 2800/2835; G01N 2800/2814; G01N 33/5088

(57) ABSTRACT

Treatments for synucleinopathy including Parkinson's Disease (PD), Multiple System Atrophy (MSA), and Dementia with Lewy Bodies (DLB) are largely unavailable. The invention provides methods for treating, preventing, inhibiting, and reversing synucleinopathy by attenuating MSH activity, decreasing MSH expression, or by modulating MSH engagement with its receptor by utilizing antagonists. Furthermore, the inventor has provided a method for producing a synucleinopathy animal model for screening treatments and for studying synuclein disease pathology.

### CROSS-REFERENCE TO RELATED APPLICATIONS

Not Applicable.

### STATEMENT REGARDING FEDERALLY SPONSORED RESEARCH OR DEVELOPMENT

Not Applicable.

### BACKGROUND OF THE INVENTION

### 1. Field of the Invention

The present invention relates generally to synuclein diseases and, in particular, methods for producing animal disease models for synucleinopathy, and methods for preventing, inhibiting, and reversing synuclein diseases.

### 2. Description of Related Art

Synuclein neurogenerative diseases (sometimes referred to as synucleinopathy) are distinctly characterized by abnormal degradation-resistant accumulations or aggregations of the α-synuclein protein in protein inclusions known as Lewy bodies or glial cytoplasmic inclusions (GCI). Parkinson's Disease (PD), Multiple System Atrophy (MSA) and Dementia with Lewy Bodies (DLB) are examples of synuclein diseases. In addition to abnormal aggregates of α-synuclein, cardinal traits are the progressive loss of dopamine transmission and decrease in neuromelanin in the substantia nigra of the basal ganglia along with loss of dopaminergic neurons. The nigrostriatal dopaminergic pathway, which encompasses the substantia nigra to the dorsal striatum is integral for motor control. Motor deficits (symptoms of the disease affecting movement) and non-motor deficits (other health problems related to the disease: mood disorders, cognitive changes, hallucinations and delusions, orthostatic hypotension, sleep disorders, constipation, pain, fatigue, vision problems, urinary urgency, incontinence, loss of smell, sexual problems, weight loss, impulsive control disorder) in synucleinopathy result from diminished levels of dopamine and loss of neurons. Another hallmark of PD and MSA is dysregulation in cellular autophagy, as noted by Lynch-Day and colleagues, the various pathogeneses of the disease “are tightly linked to autophagy, a highly conserved cellular homeostatic process essential for bulk degradation of cytoplasmic contents” (Lynch-Day, 2012). Several notable differences occur in the pathogenesis of MSA compared to PD, including involvement of not only the nigrostriatal dopaminergic pathway but also the cerebellar afferent pathways in MSA. Additionally, cytoplasmic α-synuclein inclusions in MSA are found not predominantly in neurons, but in oligodendrocytes.

Symptoms of synucleinopathy such as PD include tremor, rigidity, postural instability, and bradykinesia (slowness of movement). Collectively the clinical features are known as “Parkinsonism” (Dickson, 2012). Patients afflicted with MSA display many of the same symptoms as patients with PD, though symptoms progress more rapidly and severely, leading to a higher degree of impairment than in PD (Flabeau, 2010). Dementia may also occur in PD with increased deposition of Lewy bodies with progressive formation of nondegradable α-synuclein protein in neurons. In MSA, protein deposits form in glial cells and cognitive symptoms may manifest in forms of clinical depression.

Early and sensitive biomarkers are required for the diagnosis of PD and MSA. Though several genes such as SNCA (PARK 1-4), LRRK2 (PARKS), Parkin (PARK2), PINK1 (PARK6), DJ-1 (PARK7), and ATP13A2 (PARK9), and environmental factors including exposure to pesticides, metals, and solvents have been identified as risk factors, none have provided diagnosis before emergence of Parkinsonian manifestation (Klein, 2012). Often, when cardinal symptoms are identified, the disease has already progressed, and a significant number of dopaminergic neurons are already affected (Sharma, 2013). As stated by Miller (Miller, 2015), “Current therapies treat these symptoms by replacing or boosting existing dopamine. All current interventions have limited therapeutic benefit for disease progression because damage likely has progressed over an estimated period of ˜5 to 15 years to a loss of 60%-80% of the nigral dopamine neurons, before symptoms emerge.”

Currently, there is no cure for synuclein diseases, only therapeutics targeted towards minimizing symptoms. The gold standard drug presently for PD patients is L-3,4-dihydroxyphenylalanine (L-dopa, marketed as Levodopa), a precursor of dopamine that restores dopamine levels and permits dopaminergic transmission resulting in alleviation of parkinsonism such as improved motor skills and decreased tremor and/or rigidity. However, when L-dopa levels decline or when treatment is halted, the symptoms reemerge. Parkinsonism in MSA patients is generally not alleviated by L-dopa treatment and additionally, any effect of the therapeutic dwindles over a period of two to three years. Dopamine agonists such as Pramipexole are also utilized for treatment of both PD and MSA, though L-dopa is still considered the gold standard for alleviating motor-related symptoms. Acting directly on dopamine receptors and mimicking endogenous dopamine, dopamine agonists exert anti-parkinsonian effects without cytotoxic free radical formation linked with the metabolism of dopamine (Brooks, 2000). Like L-dopa, dopamine agonists have not showed any evidence of inhibiting disease progression. Thus, further development of therapeutics that prevent, inhibit, and reverse disease progression is required.

Animal models are critical tools for studying the pathogenesis of synucleinopathy and for screening in vivo effects of potential therapeutics. Current animal models are classified under toxin-based models, using toxins such as MPTP (1-methyl-4-phenyl-1,2,3,6-tetrahydropyridine) as an inducer, and genetic models based on identified PD-related genes. While these models have had a tremendous impact in the current understanding of dopaminergic neuronal biology and synuclein pathology, and have been used extensively in drug development for synucleinopathy, both toxins and genetic models may only model some aspects of the disease and do not accurately represent the human disease or embody crucial pathological features of disease progression. For example, toxins such as MPTP and 6-OHDA (6-hydroxydopamine), while able to damage dopaminergic neurons that result in behavioral motor deficits, Lewy bodies have not been observed (Blesa, 2014), which is a hallmark in synuclein pathology. Genetic models provide a promising alternative; however, only a small percentage of patients with PD or MSA ever show defects in those PD-related genes. A model that recapitulates synuclein pathology, disease onset and progression of neurodegeneration at the cellular level in vitro and in vivo and at the behavioral level in animals is still needed.

### BRIEF SUMMARY OF THE INVENTION

Accordingly, it is an object of the invention to overcome these and other problems associated with the related art. These and other objects, features and technical advantages are achieved by methods for preparing an animal model of synucleinopathy, and by methods for treating, preventing, inhibiting, and reversing synuclein diseases.

This invention provides methods for inducing synucleinopathy including Parkinson's Disease (PD), Multiple System Atrophy (MSA), and Dementia with Lewy bodies (DLB) in non-human animal models, including mice, rats and non-human primates (NHP), comprising administering Melanocyte Stimulating Hormone (MSH), including α-Melanocyte Stimulating Hormone (α-MSH) or β-Melanocyte Stimulating Hormone (β-MSH), and generally termed MSH, where administration of MSH may be through an intranasal route, through injection to the animal brain, or through a stereotaxic method.

This invention provides methods for screening a molecule for therapeutic use for treatment of synucleinopathy by providing an animal having synucleinopathy by administering to the animal MSH, measuring in the brain accumulation of α-synuclein, aggregation of α-synuclein, loss of autophagy, loss of dopaminergic neuronal pigmentation, loss of cell viability, and measuring loss of motor control, administering to the animal the molecule to be screened for therapeutic use; and selecting the molecule as a therapeutic drug if at least one of accumulation of α-synuclein, aggregation of α-synuclein, loss of autophagy, loss of dopaminergic neuronal pigmentation, loss of cell viability, and loss of motor control is decreased.

This invention provides a method for treating, preventing, inhibiting or reversing synucleinopathy comprising attenuated binding of MSH to its receptor. The receptor may be, but is not limited to, Melanocortin 1 Receptor (MC1R). The synucleinopathy is selected from the group consisting of PD, MSA and DLB. In some aspects, the compound may attenuate binding of MSH to its receptor by engaging the receptor whereby MSH is blocked from engaging the receptor. In other aspects, the compound may block MSH from engaging with the receptor and may additionally be an inverse agonist, thereby reversing the effects of MSH upon engagement with the receptor. The compound may be a protein selected from the group consisting of ASIP, AgRP, and HBD3. The compound may be an antibody or nanobody. The compound may be a protein mutant, protein variant, or peptide fragment of a protein, such that the protein mutant, variant or peptide fragment reverses MSH activity and/or blocks MSH from binding to its receptor by engaging the receptor. The protein mutant, variant, or peptide fragment may be a mutant, variant, or peptide fragment of a protein selected from the group including ASIP, AgRP, and HBD3. In some aspects the compound used to attenuate or reverse MSH activity may be combined with an antibody targeting the human transferrin receptor. The compound may be fused to a human transferrin receptor specific antibody or the compound may be bound by an antibody fused to a human transferrin. The compound may be a naturally occurring or synthesized small molecule of less than 1000 dalton.

This invention provides a method for treating, preventing, inhibiting or reversing synucleinopathy comprising modulating the function of MSH transcripts. In some aspects, the synucleinopathy is selected from a group including PD, MSA and DLB. Preferably, the function of MSH transcripts is modulated by contacting an MSH nucleic acid with a compound such that expression of MSH is inhibited. In accordance with an aspect of the invention, the compound is selected from the group consisting of an antisense nucleic acid, a ribozyme, a triplex-forming oligonucleotide, a siRNA, a primer, and any combination thereof. The antisense nucleic acid can be selected from the group consisting of an oligonucleotide, an antisense oligonucleotide, a DNA oligonucleotide, an RNA oligonucleotide, an RNA oligonucleotide having at least a portion of said RNA oligonucleotide capable of hybridizing with RNA to form an oligonucleotide-RNA duplex, and a chimeric oligonucleotide.

Preferably, the compound is a nucleic acid targeted to a nucleic acid molecule encoding MSH, wherein said compound hybridizes with said nucleic acid molecule encoding MSH and inhibits the expression of an MSH protein. In accordance with an aspect of the invention, the compound is an oligonucleotide. In an alternative, the compound is an antisense oligonucleotide. In another alternative, the compound is a DNA oligonucleotide. In yet another alternative, the compound is an RNA oligonucleotide. The compound may also be a chimeric oligonucleotide. In accordance with another aspect of the invention, at least a portion of said compound hybridizes with RNA to form an oligonucleotide-RNA duplex. In accordance with another aspect of the invention, the compound has at least one modified internucleoside linkage, sugar moiety, or nucleobase.

In another alternative, the compound is a siRNA. The MSH specific siRNA may be directed to an MSH transcript, or any combination thereof. The siRNA may comprise RNA lacking stable internal repeats or may comprise hairpin RNA.

In yet another aspect of the invention, the gene function of MSH is altered by introducing into cells containing MSH nucleic acids an engineered non-naturally occurring Clustered Regularly Interspaced Short Palindromic Repeats (CRISPR)-CRISPR associated (Cas) (CRISPR-Cas) system.

In yet another aspect of the invention, it may also exist whereby there is a means to analyze the effect of MSH on α-synuclein expression. There are a multitude of ways to perform this analysis. Immunofluorescence or immunohistochemical methods may be employed, with a custom software program (e.g. ImageJ, MATLAB) used to detect α-synuclein from images. The images are uploaded to the specific software program and the images are analyzed for α-synuclein. The difference in α-synuclein expression in images can then be compared. For instance, regarding an animal model, the images of brain explants exposed to α-MSH or β-MSH for 1 h, 24 h, 48 h and other time points can be compared to study the effect of α-MSH or β-MSH exposure time on α-synuclein expression. Similarly, if a blocker for α-MSH or β-MSH receptors is exposed to brain explants, the effect of the blocker on MSH's ability to modulate α-synuclein expression can be studied. Other methods to study α-synuclein expression include, but are not limited to: ELISA, flow cytometry, PCR, spectrophotometry, and cell and protein-based assays (BCA, Alamar Blue). In any of these techniques, the effect of MSH or the blockage of MSH receptors on α-synuclein expression can be studied.

These and other features, aspects and advantages of the present invention will become better understood with reference to the following description, examples and appended claims.

### BRIEF DESCRIPTION OF THE SEVERAL VIEWS OF THE DRAWINGS

FIG. 1A is a bar chart depicting α-MSH disabling autophagy in MNT-1 cells when autophagy is induced by rapamycin. Cellular autophagy induced by rapamycin is reduced upon exposure to α-MSH resulting in lower melanin levels, whereas, an increase in melanin content is observed in untreated cells.

FIG. 1B is a photograph of cells in wells of a culture plate depicting starvation-induced autophagy in cells is inhibited by α-MSH resulting in inhibition of melanin production. Melanin formation (dark pigmentation) is observed in untreated starved cells exhibiting normal cellular autophagy (left well), while melanin formation is not observed in α-MSH treated starved cells exhibiting disabled cellular autophagy (right well).

FIGS. 2A-C are immunofluorescence microscopy photographs depicting α-MSH inducing accumulation and aggregation of α-synuclein indicated by the arrows (FIG. 2B), and ASIP inhibition of α-MSH induced accumulation of α-synuclein (FIG. 2C).

FIG. 3 is a bar chart and related photographs of plates depicting ASIP reversal of α-MSH induced block in cellular autophagy; ASIP, acting as inverse agonist, reverses α-MSH effects in exposed cells and restores cellular autophagy as indicated by the increase in melanin content versus the α-MSH exposed cells not treated with ASIP, which remained unable to form melanin.

FIGS. 4A-C depict the mechanism of cell death induced by α-MSH and inhibition by ASIP, which blocks cell death. FIG. 4A is a graph depicting cells in culture that when exposed to α-MSH do not slow down metabolism to adjust to low nutrient conditions so that glucose consumption is observed to be faster in α-MSH exposed cells as compared to cells not exposed to α-MSH. FIG. 4B (Left 2 panels) are fluorescence microscopy photographs of α-MSH exposed cells (6 days) that die by apoptosis (bottom-left panel) due to, in part, a depleted nutrient supply (e.g., inadequate glucose levels, FIG. 4a). Vulnerable cells appear with higher fluorescence intensity for the marker of apoptosis, while untreated cells remain alive and show only background fluorescence (top-left panel). The third panel to the right are graphs of flow cytometry plots of average fluorescence intensity for the marker of apoptosis after 4, 7 and 9 days of exposure to α-MSH (green line) or medium alone (blue filled), showing increasing apoptotic events in α-MSH treated cells with time but not in untreated cells. FIG. 4C is a bar chart depicting ASIP blocking α-MSH effects and protecting against cell death. The arrow indicates the observation that viability is higher in α-MSH exposed cells treated with 200 nM ASIP as compared to α-MSH exposed cells without ASIP treatment.

FIGS. 5A-B depict the in vivo induction of synucleinopathy in mice by α-MSH. FIG. 5A is a drawing showing the administration of α-MSH into the left nostril of the mouse and a timeline showing frequency of administration and behavioral testing performed by rotorod. FIG. 5B is a series of rotorod testing photographs of a mouse chronically administered α-MSH by intranasal route for a period of 1 month to induce synucleinopathy (see FIG. 5a). The mouse is representative of 2 other mice, all of which exhibited progressive motor deficits. The mouse shown, exhibits normal gait 2 weeks after the end of induction of synucleinopathy. However, after 4 weeks, impaired/weakened right hind leg, loss of symmetry in gait become apparent and its center of gravity shifts inward so that its left leg compensates and moves inward (circle) on each step. Progressive decline in gait was observed at later period of 6 and 8 weeks, not shown.

FIGS. 6A-B are immunohistochemistry microscopy photographs of brain sections stained with anti-mouse alpha synuclein (BD Biosciences, Clone 42/α-Synuclein). The series of photographs (increasing magnification from left to right) depict alpha-synuclein aggregation observed in a neuron located in the left substantia nigra of mouse described in FIG. 5, which was administered α-MSH in the left nostril (FIG. 6a). No alpha-synuclein aggregation was observed in the substantia nigra neuron of mouse administered with vehicle control (no α-MSH, FIG. 6b).

FIGS. 7A-D are photographs, schematic diagrams and bar graphs that depict the characterization of myc-ASIP (FIGS. 7a and 7b) and its brain delivery vehicle, anti-myc-Tf (transferrin) (FIGS. 7c and 7d). FIG. 7A consist of a photograph from an SDS-PAGE Coomasie-blue gel showing bands of purified myc-ASIP prepared under non-reducing (Lane 1) and reducing (Lane 2) conditions, migrating at the expected distance showing ˜15 kDa band. The molecular weight of the band is based on the protein ladder marker (ThermoScientific, PAGE ruler). On the right is a schematic drawing of myc-ASIP construct. FIG. 7B is a bar chart depicting myc-ASIP blocking of α-MSH induced reduction of melanin formation by MNT-1 cells. The arrow indicates the observation that melanin level is higher in α-MSH exposed cells treated with 500 nM myc-ASIP as compared to α-MSH exposed cells without myc-ASIP treatment.

FIG. 7C consist of a photograph from an SDS-PAGE Coomasie-blue gel showing bands of purified anti-myc-Tf prepared under non-reducing (Lane 1) and reducing (Lane 2) conditions, migrating at the expected distances showing: a) the non-reduced intact anti-myc-TF antibody fusion protein (˜280 kDa band, Lane 1) and b) reduced light chain (˜25 kDa band, Lane 2) and reduced heavy chain fused to a transferrin ligand (˜115 kDa band, Lane 2), respectively. The molecular weight of the bands is based on the protein ladder marker (ThermoScientific, PAGE ruler). On the right is a schematic drawing of anti-myc-Tf construct consisting of 2 light chains+2 heavy-chains fused to a transferrin ligand with a combined molecular weight of approximately 280 kDa. FIG. 7D is a bar chart depicting the formation of a stable complex between anti-myc-Tf and myc-ASIP verified using an ELISA method.

### DETAILED DESCRIPTION OF THE INVENTION

Methods for Inducing Synucleinopathy in Non-Human Mammals for an Animal Disease Model of Synucleinopathy

The present invention is directed to a method for inducing synuclein diseases including Parkinson's Disease (PD), Multiple System Atrophy (MSA) and Dementia with Lewy body (DLB) in a non-human mammal, resulting in an animal displaying the symptoms of the synuclein disease. The animal model prepared as described herein can be utilized to study pathways leading to synucleinopathy, pathology of synucleinopathy, screen therapeutics for the treatment of synucleinopathy, and study the effects of therapeutics on cellular pathogenesis and physical symptoms.

Previous teachings (Cotzias, 1967) had implicated β-Melanocyte Stimulating Hormone, a neuropeptide hormone similar in structure and function to α-Melanocyte Stimulating Hormone, as a therapeutic to inhibit pathways leading to synucleinopathy such as PD. MSH are signaling hormones produced from proopiomelanocortin (POMC), a polypeptide hormone precursor. To exert their effects, MSHs bind to melanocortin receptors, including Melanocortin Receptor 1 (MC1R), which fall under the G protein coupled receptor (GPCR) superfamily. Upon binding, receptor engagement activates cAMP production and triggers downstream events including various metabolic pathways, genomic maintenance pathways and melanin biosynthesis. The substantia nigra (SNpc) in normal brains contain conspicuous amounts of melanin and since the substantia nigra in brains afflicted with PD showed significantly less melanin, researchers believed increasing β-MSH in the brain would stimulate melanin production in this part of the brain restoring dopamine to normal levels and ameliorate the parkinsonian symptoms (Fahn, 2015). However, treatment with β-MSH resulted in worsening of Parkinsonian symptoms, although darkenening of skin of treated patients was also observed. Researchers believed the aggravation of symptoms was due to diversion of precursors of melanin away from the brain to the darkened skin (Cotzias, 1967). The researchers did not associate or suggest that the cause of nigral degeneration in PD was due to β-MSH or identify β-MSH as a key factor in the progression of neurodegeneration in synucleinopathy.

In contrast, the present invention provides that MSH induces synucleinopathy, is a major contributor for disease progression, and can be utilized as an inducer for producing animal disease models of synucleinopathy, including PD, MSA and DLB. It is clear based on examples presented herein that the presence of α-MSH triggers synucleinopathy both on cellular levels and through physical symptoms and brain pathology accurately replicating human synucleinopathy in an animal disease model. Upon exposure to α-MSH, major hallmarks of synucleinopathy are observed, including accumulation and aggregation of α-synuclein protein, and decreased melanin. Likewise, examples presented herein show that exposure to α-MSH impairs cellular autophagy and ultimately results in cell death of vulnerable cells by apoptosis. Disabled autophagy has been shown to facilitate the buildup of protein aggregates in dopaminergic neurons (Sato, 2018). This is consistent with the observation that decreased neuromelanin in PD coincided with “autophagic degeneration” or increased number of vacuoles with cytoplasmic materials as revealed in ultrastructural examination of melanized neurons of the substantia nigra in PD patients (Anglade, 1997). There is also a reduction in autophagic proteins such as LAMP1 specifically in nigral neurons with α-syn inclusions (Chu, 2014). Carriers of mutations in genes including SNCA, GBA and LRRK2 are at a much higher risk of developing PD (Lill, 2016). Notably, these genes play important roles in cellular autophagy (Manzoni, 2013, Beilina, 2016).

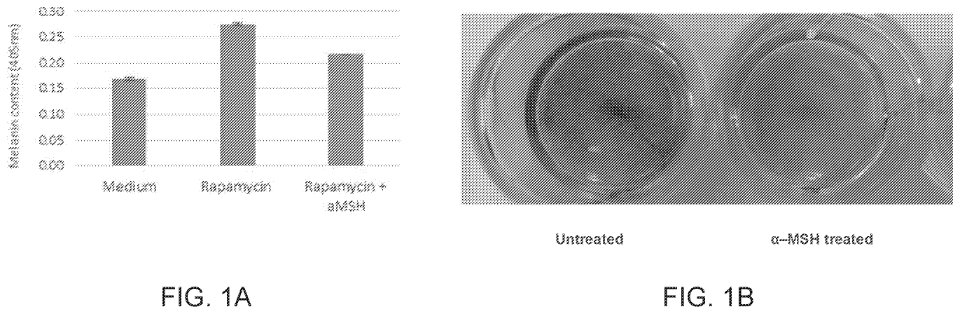

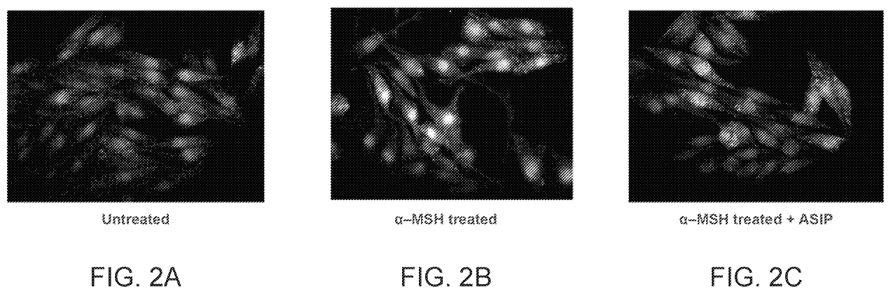

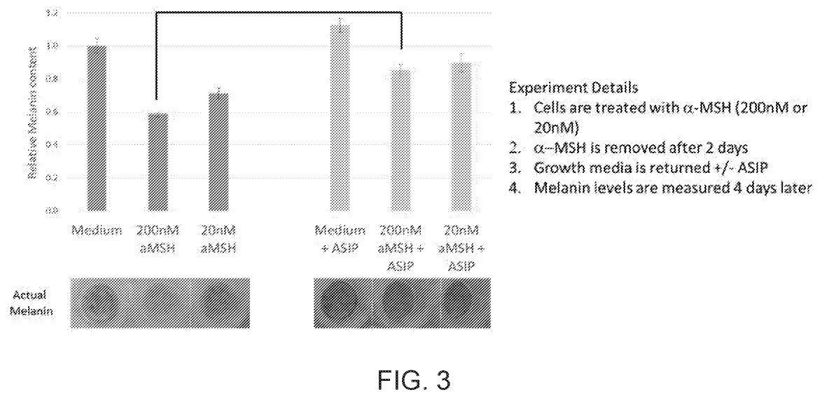

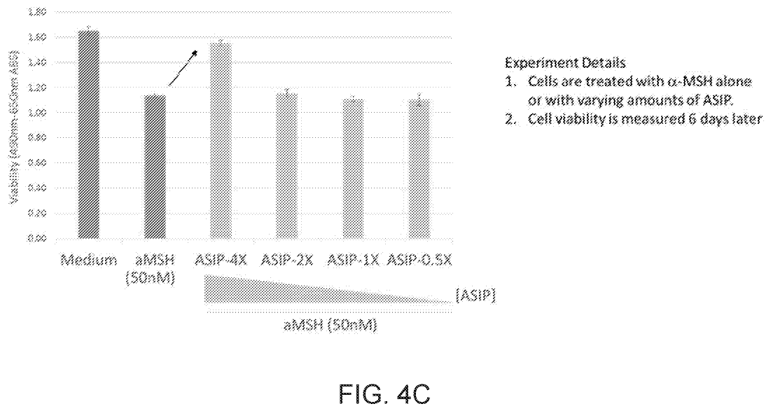

FIGS. 1-6 show the induction of synucleinopathy in vitro and in vivo upon exposure to α-MSH employing the MNT-1 cell line (a gift from Michael S. Marks, Ph.D.) as an in vitro model of neuromelanin-containing neurons and the mouse administered α-MSH by intranasal route, respectively. Upon contact with α-MSH, the human pigmented cell line MNT-1 displayed the cardinal features of synucleinopathy starting with accumulation and aggregation of α-synuclein (a major component of Lewy bodies and glial cytoplasmic inclusions in diseased cells of brains afflicted with PD, MSA and DLB) as shown by an increase in intensity of immunofluorescence staining of intracellular α-synuclein (FIG. 2B), compared to cells not exposed to α-MSH (FIG. 2A). Additionally, rapamycin induced autophagy was reduced in the prescence of α-MSH (FIG. 1A). Normally an increase in melanin production by MNT-1 is observed in the presence of rapamycin, since rapamycin induces autophagy and autophagy-related genes that promote melanogenesis. Melanin levels serve as a reliable readout of autophagy in MNT-1 (Kalie, 2013). Indeed, a decrease in melanin was observed when autophagy was inhibited in melanocytes (Zhang, 2015). Similarly, in nutrient-depleted conditions, which induces cellular autophagy and increase melanin production, exposure to α-MSH inhibited autophagy and melanin formation (FIG. 1B). Cells with impaired autophagy do not adjust to low nutrient conditions and quickly deplete already scarce essential nutrients e.g., glucose. As expected, cells exposed to α-MSH consume glucose faster than untreated cells (FIG. 4A), and as a result deplete the medium of glucose and vulnerable cells starve and die by apoptosis, as measured by a fluorescence-based method that detects activated caspases in apoptotic cells (FIG. 4B). The cellular events observed upon exposure to α-MSH, including accumulation and aggregation of α-synuclein, decreased melanin production, disruption of the normal autophagic response and cell death by apoptosis are again consistent with the pathology observed in the brains of PD and MSA (Xu, 2015; Yasuda, 2013).

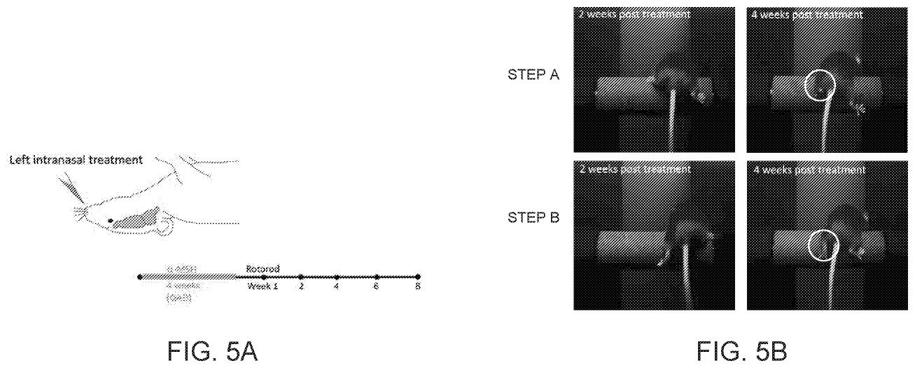

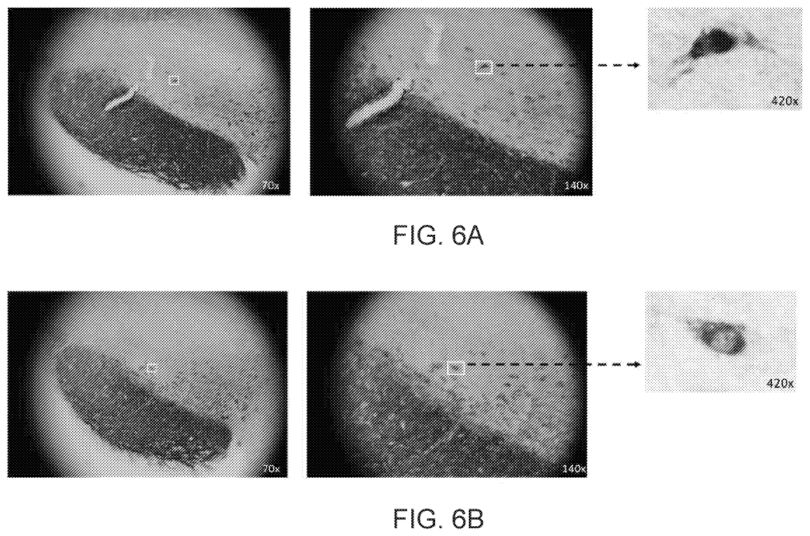

Shown by the invention described herein, animal disease models for synucleinopathy, represented by mice subjected to α-MSH, exhibited progressive impairment of gait and general weakness characteristic of synucleinopathy including PD and MSA. Three mice were given the inducer, α-MSH, which was administered once every other day for one month through an intranasal route through the left nostril (FIG. 5a), and one mouse not subjected to the α-MSH inducer served as a healthy control. Observations and measurements were taken weekly at the end of α-MSH administration. No significant gait impairment was observed after two weeks of α-MSH administration. Measurements taken four, six, and eight weeks after α-MSH administration showed progressive weakness and deteriorating motor control in the mice given α-MSH (FIG. 5b, two and four weeks shown). Loss of symmetry in gait was observed in the right hind legs of the mice, accompanied by an inward shift in the animal's center of gravity and compensation by the left leg (FIG. 5b, STEP A), which shifted inward during movement (FIG. 5b, STEP B). The control mouse not subjected to α-MSH maintained normal gait and leg movement. Gait impairment observed in the three mice given the α-MSH, is characteristic of motor symptoms associated with PD and MSA symptoms. Moreover, the time-dependent intensified serverity of gait impairment after α-MSH administration is indicative of disease progression as measured through manisfestation of worsening physical symptoms. A cardinal feature of synucleinopathy pathology is the presence of aggregated intracellular alpha-synuclein, which is the predominant constituent of Lewy bodies and glial cytoplasmic inclusions (GCIs) observed in the affected regions of the brain in PD, MSA and DLB. Immunohistological analysis of the brain of the same mouse described in FIG. 5, revealed a Lewy body-like aggregate of alpha-synuclein in the left substantia nigra (FIG. 6a), indicative of, and consistent with, synuclein disease induction after α-MSH administration. Pathology was absent in the control mouse not subjected to α-MSH (FIG. 6b).

Hence, the invention provides methods and compositions for producing animal models of synuclein diseases. Using the inducer α-MSH and/or β-MSH, animal disease models can be created to facilitate studying disease pathways, screen therapeutics for the treatment of the diseases, and study the effects of therapeutics.

In the invention presented herein, the non-human mammal is given the inducer α-Melanocyte Stimulating Hormone (α-MSH) or β-Melanocyte Stimulating Hormone (β-MSH) to induce the synuclein disease for an animal model of synucleinopathy. The inducer may be given to the animal by intranasal route, by injection to the animal's brain, or through a stereotaxic method. The α-MSH or β-MSH peptides used in this invention have the amino acid sequences of Ref seq ID numbers [α-MSH: SEQ ID NO: 12, β-MSH: SEQ ID NO: 15].

Therapeutics

Previous teachings for synucleinopathy therapeutics improve parkinsonian symptoms by replenishing dopamine but do not improve conditions for replenishing viable cellular function to stop and reverse disease progression. Thus, further development of therapeutics that prevent, inhibit, and reverse disease progression without deleterious side effects is required.

Antagonists Blocking MSH Binding to Receptors

For therapeutics, a patient suffering from a synuclein disease can be treated by admistering a molecule or compositions identified herein that can neutralize, block, reverse and/or attenuate the signaling activity of MSH. Hence, the invention herein provides several methods and compositions to facilitate the treatment, prevention, inhibition, and reversal of MSH-driven diseases (e.g., synucleinopathy) by diminishing MSH activity and related pathways.

In one non-limiting example, the methods comprise the step of administering to the patient in need of treatment, a therapeutically effective amount of an antagonist of MSH binding to a receptor. The receptor may be, but is not limited to, the Melanocortin 1 Receptor (MC1R), which is expressed by dompaminergic neurons of the substantia nigra (Chen, 2017). The antagonist described in the present invention effectively inhibits the activity of MSH protein by blocking its interaction with the receptor. The antagonist may also be an inverse agonist that is able to engage the receptor, and elicit the opposite response, thereby effectively reversing the deleterious effects of MSH.

In the cell, reduction of synuclein disease by a therapeutic may be measured by at least one of, but not limited to, the following: a decrease in α-synuclein protein accumulation, a decrease in α-synuclein protein aggregation, restoration of melanin production, and restoration of autophagy and general cell viability. In the brain, reduction of synuclein disease pathology may be measured in neurons in the substantia nigra for PD, in oligodendroglial cells in the cerebellum for MSA and cortical neurons in DLB. Return of cerebrospinal fluid α-MSH to normal levels and improvements in behavioral symptoms (e.g., akinesia, brady-/hypo-kinesia), may be measured for reversal of synucleinopathy in patients. Therefore, α-MSH is a potential synuclein disease biomarker.

In some aspects antibody or nanobody is used to block binding of MSH to its receptor, including but not limited to MC1R, to inhibit downstream pathways leading to synucleinopathy, or to reverse synucleinopathy.

In some aspects Agouti Signaling Protein (ASIP), a peptide fragment of ASIP, a mimetic of ASIP, a homolog of ASIP, or a variant of ASIP is used to block binding of MSH to its receptor, including but not limited to MC1R, to inhibit downstream pathways leading to synucleinopathy, or to reverse synucleinopathy. ASIP is an endogenous competitive antagonist of MSH binding to receptor, and is also an inverse agonist that decreases basal receptor signaling (Wolf Horrell, 2016). In some aspects therapeutics for treatment of synucleinopathy comprise homologs of ASIP, including Agouti Related protein (AgRP), a fragment of AgRP, a mimetic of AgRP, or a variant of AgRP. AgRP is an endogenous competitive antagonist and an inverse agonist of melanocortin receptors, including but not limited to the receptor MC1R (Chai, 2003). The N-terminal of AgRP facilitates antagonistic activity and regulates C-terminal antagonist activity to melanocortin receptors. The C-terminus interacts with the receptor and is an inhibitor of MSH binding to its receptor (Jackson, 2006). In some aspects, treatment of synucleinopathy includes blocking the binding of MSH at its receptor with the C-terminus of AgRP, a mimetic of the C-terminus of AgRP, a fragment of the C-terminus of AgRP, or a variant of the C-terminus of AgRP. In some aspects, human β-defensin3 (HBD3), an endogenous neutral antagonist of melanocortin receptors, including but not limited to MC1R, that blocks binding of MSH, but does not affect basal receptor signaling (no inverse agonist activity), may be used as a therapeutic for synucleinopathy. Treatment of synucleinopathy can include HBD3, a peptide fragment of HBD3, a mimetic of HBD3, or a variant of HBD3.

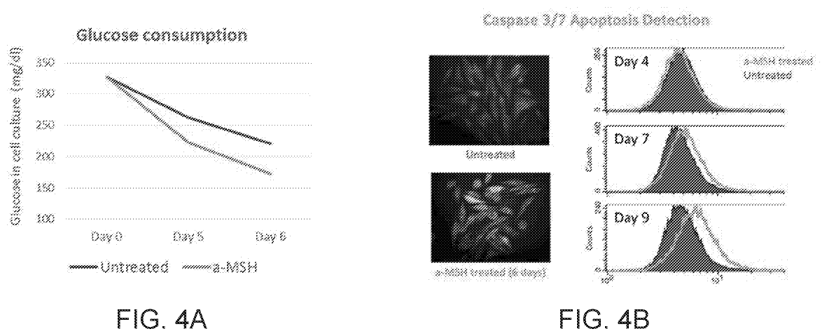

FIGS. 2C, 3, and 4C show human ASIP inhibiting the effects of α-MSH in cellular models and is an effective therapeutic for preventing and reversing synuclein pathology. ASIP was tested as a therapeutic for synucleinopathy in the in vitro cellular model of neuromelanin-containing neurons modeled by MNT-1 cells. As described herein, the cells were first exposed to α-MSH, and shortly thereafter displayed accumulation and aggregation of intracellular α-synuclein (FIG. 2B, arrows), decreased melanin production (FIG. 1B), disablement of cellular autophagy (FIG. 1A), and increased cell death by apoptosis (FIG. 4B)—all cardinal features of synucleinopathy. Following exposure to ASIP, the effects of α-MSH were blocked and reversed in the in vitro cellular model as shown by decreased levels of α-synuclein measured by immunofluorescence (FIG. 2C), restoration of melanin production (FIG. 3) and protection against cell death (FIG. 4C). Immunofluorescence from the α-MSH and ASIP treated cells display similar quantity of α-synuclein compared to healthy, non-treated cells. This indicates ASIP inhibits the accumulation and aggregation of intracellular α-synuclein, and additionally has the potential to reverse the accrual of toxic protein aggregates and reverse impairment of autophagy. Furthermore, cells that showed depleted melanin production upon exposure to α-MSH demonstrated restoration of melanin levels following treatment with ASIP (FIG. 3). ASIP also protects against cell death, blocking deleterious effects of α-MSH. Cells maintain viability when exposed to α-MSH when ASIP is also present in the media (FIG. 4C). Thus, as a therapeutic ASIP promotes an environment ideal for recovery and is amenable for cell restoration strategies. Based on the in vitro cellular model presented herein, ASIP halts induction and progression of synucleinopathy and reverses the disease.

In some aspects, the invention utilizes a purified protein or peptide. The protein or peptide may be ASIP, AgRP, or HBD3. Variants of native ASIP, AgRP, HBD3 proteins such as fragments, analogs and derivatives of ASIP, AgRP, and HBD3 are also within the invention. The ASIP, AgRP, HBD3 protein variants may have peptide sequences that differ from the native proteins in one or more amino acids. The peptide sequence of such variants can feature a deletion, addition, or substitution of one or more amino acids of native ASIP, AgRP, HBD3. The variant proteins substantially maintain a native protein functional activity, functional activity being binding, interacting, or engaging with the receptor, including but not limited to the MC1R receptor.

ASIP, AgRP, HBD3 or variants of the proteins corresponding to one or more particular motifs and/or domains domains or to arbitrary sizes are intended to be within the scope of the present invention. Isolated peptidyl portions of ASIP, AgRP, HBD3 proteins can be obtained by screening peptides recombinantly produced from the corresponding fragment of the nucleic acid encoding such peptides. In addition, fragments can be chemically synthesized using techniques known in the art such as conventional Merrifield solid phase f-Moc or t-Boc chemistry. For example, ASIP protein of the present invention may be arbitrarily divided into fragments of desired length with no overlap of the fragments, or preferably divided into overlapping fragments of a desired length. The fragments can be produced (recombinantly or by chemical synthesis) and tested to identify those peptidyl fragments which can function as antagonists of α-MSH binding to its receptor, including but not limited to MC1R.

Another aspect of the present invention concerns recombinant forms of the ASIP, AgRP, and HBD3 proteins. Recombinant proteins preferred by the present invention in addition to native proteins, are encoded by a nucleic acid that has at least 25% sequence identity with the nucleic acid sequence of Ref seq ID numbers [ASIP: SEQ ID NO: 2, AgRP: SEQ ID NO: 5, HBD3: SEQ ID NO: 8]. In a preferred embodiment, variant proteins prevent or attenuate MSH binding to melanocortin receptors, including but not limited to MC1R.

ASIP, AgRP, and HBD3 protein variants can be generated through various techniques known in the art. For example, ASIP protein variants can be made by mutagenesis, such as by introducing discrete point mutation(s), or by truncation. Mutation can give rise to a protein variant having substantially the same, or merely a subset of the functional activity of protein. Alternatively, forms of the protein can be generated which enhance the antagonistic function of the naturally occurring form of the protein, such as by lowering the dissociation constant (K_{d}) of binding to receptors, for instance MC1R. Other variants of ASIP, AgRP, and HBD3 proteins can be generated to include those that are resistant to proteolytic cleavage, as for example, due to mutations which alter protease target sequences.

As another example, ASIP, AgRP, and HBD3 protein variants can be generated from a degenerate oligonucleotide sequence. Chemical synthesis of a degenerate gene sequence can be carried out in an automatic DNA synthesizer, and the synthetic genes then ligated into an appropriate expression vector. One purpose for a degenerate set of genes is to provide, in one mixture, all of the sequences encoding the desired set of potential protein sequences. The synthesis of degenerate oligonucleotides is well known in the art (see, e.g., Narang, S A (1983) Tetrahedron 39:3; Itakura et al. (1981) RECOMBINANT DNA, PROC 3RD CLEVELAND SYMPOS. MACROMOLECULES, ed. AG Walton, Amsterdam: Elsevier pp 273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins (see, e.g., Scott et al. (1990) Science 249:386-390; Roberts et al. (1992) Proc. Natl. Acad. Sci. USA 89:2429-2433; Devlin et al. (1990) Science 249: 404-406; Cwirla et al. (1990) Proc. Natl. Acad. Sci. USA 87: 6378-6382; as well as U.S. Pat. Nos. 5,223,409; 5,198,346; and 5,096,815).

Similarly, a library of coding sequence fragments can be provided for an ASIP, AgRP, and HBD3 gene clone in order to generate a variegated population of ASIP, AgRP, and HBD3 protein fragments for screening and subsequent selection of fragments having one or more native ASIP, AgRP, and HBD3 binding activities to receptors, including but not limited to the receptor MC1R. A variety of techniques are known in the art for generating such libraries, including chemical synthesis. In one embodiment, a library of coding sequence fragments can be generated by (i) treating a double-stranded PCR fragment of an ASIP, AgRP, and HBD3 gene coding sequence with a nuclease under conditions wherein nicking occurs only about once per molecule; (ii) denaturing the double-stranded DNA; (iii) renaturing the DNA to form double-stranded DNA which can include sense/antisense pairs from different nicked products; (iv) removing single-stranded portions from reformed duplexes by treatment with SI nuclease; and (v) ligating the resulting fragment library into an expression vector. By this exemplary method, an expression library can be derived which codes for N-terminal, C-terminal and internal fragments of various sizes.

A wide range of techniques are known in the art for screening gene products of combinatorial libraries made by point mutations or truncation, and for screening cDNA libraries for gene products having a certain property. Such techniques will be generally adaptable for rapid screening of the gene libraries generated by the combinatorial mutagenesis of ASIP, AgRP, and HBD3 gene variants. The most widely used techniques for screening large gene libraries typically involve cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected.

The invention also provides for generation of ASIP, AgRP, and HBD3 mimetics, e.g. peptide or non-peptide agents, that are able to disrupt binding or interaction of of MSH to receptors, including but not limited to MC1R. Thus, the techniques described herein can also be used to map which determinants of ASIP, AgRP, and HBD3 participate in the intermolecular interactions involved in blocking MSH binding to receptors. The critical residues of ASIP, AgRP, and HBD3 involved in molecular recognition of the receptors can be determined and used to generate ASIP, AgRP, and HBD3 protein-derived peptidomimetics which competitively inhibit binding of MSH to receptors. By employing scanning mutagenesis to map the amino acid residues of ASIP, AgRP, and HBD3 that are involved in binding, peptidomimetic compounds can be generated which mimic those residues of a native ASIP, AgRP, and HBD3 protein. Such mimetics may then be used to interfere with the normal function of MSH signaling pathways upon binding to receptors, which include, but are not limited to MC1R.

For example, non-hydrolyzable peptide analogs of such residues can be generated using benzodiazepine (see, e.g., Freidinger et al. in Peptides: Chemistry and Biology, G. R. Marshall ed., ESCOM Publisher: Leiden, Netherlands, 1988), azepine (e.g., see Huffman et al. in Peptides: Chemistry and Biology, G. R. Marshall ed., ESCOM Publisher: Leiden, Netherlands, 1988), substituted gamma lactam rings (Garvey et al. in Peptides: Chemistry and Biology, G. R. Marshall ed., ESCOM Publisher: Leiden, Netherlands, 1988), keto-methylene pseudopepitides (Ewenson et al. (1986) J. Med. Chem. 29:295; and Ewenson et al. in Peptides: Structure and Function (Proceedings of the 9th American Peptide Symposium) Pierce Chemical Co. Rockland, III., 1985), beta-turn dipeptide cores (Nagai et al. (1985) Tetrahedron Lett 26:647; and Sato et al. (1986) J. Chem. Soc. Perkin. Trans. 1: 1231), and beta-aminoalcohols (Gordon et al. (1985) Biochem. Biophys. Res. Commun. 126:419; and Dann et al. (1986) Biochem. Biophys. Res. Commun. 134:71). ASIP, AgRP, and HBD3 proteins may also be chemically modified to create ASIP, AgRP, and HBD3 protein derivatives by forming covalent or aggregate conjugates with other chemical moieties, such as glycosyl groups, lipids, phosphate, acetyl groups and the like. Covalent derivatives of ASIP, AgRP, and HBD3 protein can be prepared by linking the chemical moieties to functional groups on amino acid side chains of the protein or at the N-terminus or at the C-terminus of the polypeptide.

The present invention further pertains to methods of producing the subject ASIP, AgRP, and HBD3 proteins. For example, a host cell transfected with a nucleic acid vector directing expression of a nucleotide sequence encoding the subject polypeptides can be cultured under appropriate conditions to allow expression of the polypeptide to occur. The cells may be harvested, lysed, and the protein isolated. A recombinant ASIP, AgRP, and HBD3 protein can be isolated from host cells using techniques known in the art for purifying proteins including ion-exchange chromatography, gel filtration chromatography, ultrafiltration, electrophoresis, and immunoaffinity purification with antibodies specific for such protein.

For example, after an ASIP, AgRP, and HBD3 protein has been expressed in a cell, it can be isolated using any immuno-affinity chromatography. More specifically, an anti ASIP, AgRP, and HBD3 antibody can be immobilized on a column chromatography matrix, and the matrix can be used for immuno-affinity chromatography to purify the ASIP, AgRP, and HBD3 protein from cell lysates by standard methods (see, e.g., Ausubel et al., supra). After immuno-affinity chromatography, the ASIP, AgRP, and HBD3 protein can be further purified by other standard techniques, e.g., high performance liquid chromatography (see, e.g., Fisher, Laboratory Techniques In Biochemistry And Molecular Biology, Work and Burdon, eds., Elsevier, 1980). In another embodiment, an ASIP, AgRP, and HBD3 is expressed as a fusion protein containing an affinity tag (e.g., GST, c-myc) that facilitates its purification.

Screening for Compounds that are Antagonists for MSH Binding to Receptors

The invention also encompasses methods for identifying compounds that specifically bind to receptors, including but not limited to MC1R, to block MSH binding. One such method involves the steps of providing an immobilized purified receptor or several receptors, or variants of receptors, one of which may be the MC1R protein or a variant of MC1R protein, and at least one test compound; contacting the immobilized protein or proteins with the test compound; washing away substances not bound to the immobilized protein(s); and detecting whether or not the test compound is bound to the immobilized protein(s). Those compounds remaining bound to the immobilized protein(s) are those that specifically interact with the receptor(s).

Nucleic Acids Encoding ASIP, AgRP, or HBD3 Proteins

Preferred nucleic acid molecules for use in the invention are the native ASIP, AgRP, and HBD3 polynucleotides shown herein as Ref seq ID numbers [ASIP: SEQ ID NO: 2, AgRP: SEQ ID NO: 5, HBD3: SEQ ID NO: 8]. Another nucleic acid that can be used in various aspects of the invention includes a purified nucleic acid or polynucleotide that encodes a polypeptide having the amino acid sequences of Ref seq ID numbers [ASIP: SEQ ID NO: 3, AgRP: SEQ ID NO: 6, HBD3: SEQ ID NO: 9]. In some aspects, the native ASIP, AgRP, and HBD3 polynucleotides shown herein as Ref seq ID numbers [ASIP: SEQ ID NO: 1, AgRP: SEQ ID NO: 4, HBD3: SEQ ID NO: 7] and containing upstream and downstream sequences of the encoded polypeptides may be utilized.

Nucleic acid molecules utilized in the present invention may be in the form of RNA or in the form of DNA (e.g., cDNA, genomic DNA, and synthetic DNA). The DNA may be double-stranded or single-stranded, and if single-stranded may be the coding (sense) strand or non-coding (anti-sense) strand. The coding sequence which encodes a native ASIP, AgRP, or HBD3 protein may be identical to the nucleotide sequence of Ref seq ID numbers [ASIP: SEQ ID NO: 2, AgRP: SEQ ID NO: 5, HBD3: SEQ ID NO: 8] or it may also be a different coding sequence which, as a result of the redundancy or degeneracy of the genetic code, encodes the same polypeptide as the polynucleotides of SEQ ID NO: Ref seq ID numbers [ASIP: SEQ ID NO: 2, AgRP: SEQ ID NO: 5, HBD3: SEQ ID NO: 8].

Other nucleic acid molecules intended to be within the scope of the present invention include variants of the native ASIP, AgRP, and HBD3 gene such as those that encode fragments, analogs and derivatives of a native ASIP, AgRP, and HBD3 protein. Such variants may be, e.g., a naturally occurring allelic variant of the native ASIP, AgRP, and HBD3 gene, a homolog of the native ASIP, AgRP, and HBD3 gene, or a non-naturally occurring variant of the native ASIP, AgRP, and HBD3 gene. These variants have a nucleotide sequence that differs from the native ASIP, AgRP, and HBD3 gene in one or more bases. For example, the nucleotide sequence of such variants can feature a deletion, addition, or substitution of one or more nucleotides of the ASIP, AgRP, and HBD3 gene.

In other applications, variant ASIP, AgRP, or HBD3 proteins displaying substantial changes in structure can be generated by making nucleotide substitutions that cause less than conservative changes in the encoded polypeptide. Examples of such nucleotide substitutions, are those that cause changes in (a) the structure of the polypeptide backbone; (b) the charge or hydrophobicity of the polypeptide; or (c) the bulk of an amino acid side chain. Nucleotide substitutions generally expected to produce the greatest changes in protein properties are those that cause non-conservative changes in codons. Examples of codon changes that are likely to cause major changes in protein structure are those that cause substitution of (a) a hydrophilic residue, e.g., serine or threonine, for (or by) a hydrophobic residue, e.g., leucine, isoleucine, phenylalanine, valine or alanine; (b) a cysteine or proline for (or by) any other residue; (c) a residue having an electropositive side chain, e.g., lysine, arginine, or histadine, for (or by) an electronegative residue, e.g., glutamine or aspartine; or (d) a residue having a bulky side chain, e.g., phenylalanine, for (or by) one not having a side chain, e.g., glycine.

Nucleic acid molecules encoding ASIP, AgRP, or HBD3 or heteromultimer fusion proteins are also within the invention. Such nucleic acids can be made by preparing a construct (e.g., an expression vector) that expresses an ASIP, AgRP, or HBD3 fusion protein when introduced into a suitable host. For example, such a construct can be made by ligating a first polynucleotide encoding an ASIP, AgRP, or HBD3 protein fused in frame with a second polynucleotide encoding another protein or peptide such that expression of the construct in a suitable expression system yields a fusion protein.

Delivery of Therapeutics Through the Blood Brain Barrier

For drug delivery of large biomolecules aross the Blood Brain Barrier (BBB), biomolecules can be fused to or bound by an antibody or antibody variant that crosses the BBB through an endogenous receptor mediated system. The transferrin (Tf) receptor is a transcytosis system that allows delivery of molecules from the bloodstream to the brain, and Tf receptor-antibody systems have been utilized to transport biomolecules across the BBB, similar to the transcytosis of Tf through the brain capillary endothelial barrier for iron transport. ASIP is not expected to readily cross the BBB and gain access to the brain parenchyma (Kastin, 2000; Patel, 2010). ASIP may be linked to a human Tf receptor antibody as a fusion protein that enables passage of ASIP through the blood brain barrier to reach the brain parenchyma using Tf receptors on the vascular endothelium of the brain (Pardridge, 2015).

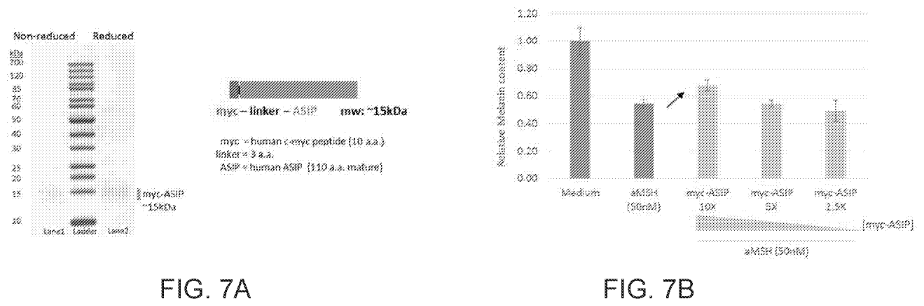

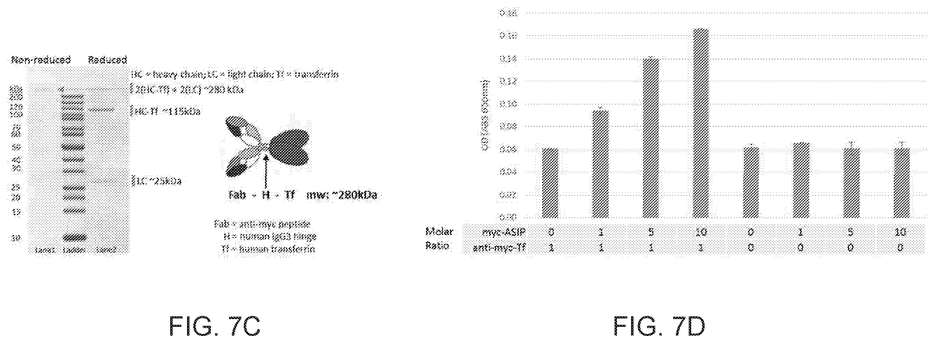

Alternatively, ASIP may be genetically fused to or bound by an antibody that has been engineered to contain a ligand (e.g., transferrin or insulin-growth factor) that enables the passage of the ASIP-antibody-ligand complex through the blood brain barrier to reach the brain parenchyma using ligand receptors on the vascular endothelium of the brain (Pardridge, 2015 and Shin, 1995). Unlike other vehicles that employ anti-Tf receptor antibodies (e.g., OX26 anti-Tf receptor) to target the BBB Tf receptor, Tf-antibody fusion protein engages the Tf receptor as a natural ligand (Shin, 1995). This feature allows it to efficiently transcytose across the BBB and reach the brain parenchyma without hindrance (Shin, 1995), whereas high affinity anti-Tf receptor antibodies are retained in the brain capillaries, resulting in suboptimal penetration of the brain parenchyma (Paterson, 2016, Shin, 1995). The Tf-antibody fusion may be engineered so that it binds ASIP directly or a tag on ASIP. The construction, purification and in vitro characterization of myc-tagged ASIP and its delivery vehicle, anti-myc-Tf are described below. In the clinic, a patient with synucleinopathy may be administered a mixture of anti-myc-Tf and myc-ASIP by intravenous injection at a dose and frequency that will ensure adequate amounts of myc-ASIP are delivered to the brain. The amount of myc-ASIP in the brain could be monitored by measuring the amount of anti-myc-TF-myc-ASIP complexes in the cerebrospinal fluid at a time after administration. An enzyme-linked immunosorbent assay (ELISA) method can be used for this purpose such as the one used and described below to measure anti-myc-TF-myc-ASIP complexes in solution (FIG. 7d).

To contruct the myc-tagged ASIP protein (myc-ASIP), myc peptide (400-419 amino acid of the human c-myc protein) was fused to the N-terminal end of human ASIP (mature sequence) using a 3 amino acid linker (FIG. 7a). The expression vector containing the myc-linker-ASIP sequence was transfected into the human 293T cell line by calcium phosphate method. A stable transfectant secreting myc-ASIP was isolated and subcloned by limiting dilution and expanded in growth medium in roller bottles. Harvested supernatant was passed through an anti-myc (clone 9E10) resin column. Bound myc-ASIP protein was eluted using a glycine buffered solution (pH2.5) and neutralized to pH-7.3 using 1M Tris. SDS-PAGE analysis of phosphate buffered saline (1×PBS) dialyzed myc-ASIP was performed under non-reducing and reducing conditions and showed the expected band of ˜15 kDa (FIG. 7a). Purified myc-ASIP exhibited activity by inhibiting α-MSH induced reduction of melanin formation by MNT-1 in a concentration-dependent manner (FIG. 7b). Anti-myc-Tf was constructed by replacing the heavy chain and light chain variable region sequences of the human anti-dansyl Tf-antibody fusion protein, which does not have the CH2 and CH3 Fc domains (Shin, 1995), with variable region sequences of anti-myc antibody clone 9E10 (Schiweck, 1997). The mouse myeloma cell line P3X63Ag8.653 was co-transfected with the modified constructs by electroporation. A stable transfectant secreting anti-myc-Tf was isolated and subcloned by limiting dilution and expanded in growth medium in roller bottles. Harvested supernatant was passed through an anti-human IgG3 hinge (clone HP6050) resin column. Bound anti-myc-Tf was eluted using a glycine buffered solution (pH2.5) and neutralized to pH-7.3 using 1M Tris. SDS-PAGE analysis of 1×PBS dialyzed anti-myc-Tf was performed under non-reducing and reducing conditions and showed the expected bands: Non-reduced: ˜280 kDa (full protein); Reduced: ˜115 kDa (heavy chain fused to Tf) and ˜25 kDa (light chain) (FIG. 7c). The ability of anti-myc-Tf to form a stable complex with myc-ASIP was tested by an ELISA method. Briefly, wells of a 96-well flat bottom plate (Immulon2 HB) was coated with anti-human ASIP (Thermofisher) and non-specific sites blocked using 3% bovine serum albumin in 1×PBS. Mixtures of anti-myc-Tf and myc-ASIP was prepared and incubated overnight in 1×PBS such that anti-myc-Tf concentration of 1 μg/ml was kept constant and myc-ASIP concentration was adjusted to reflect the molar ratio (anti-myc-Tf: myc-ASIP) of 1:1, 1:5, 1:10. Mixtures containing no anti-myc-Tf or no myc-ASIP or no proteins (i.e., 1×PBS alone) was included as negative controls. The mixtures were added to wells in replicate of 2 and the plate allowed to incubate at room temperature. Complexes of anti-myc-Tf+myc-ASIP bound by anti-ASIP capture antibody in each well were detected using a biotin-labeled anti-human Tf (Thermofisher) and streptavidin-HRP. TMB-substrate (Biolegend) was added and absorbance (OD650 nm) measured using a plate reader (Molecular Devices). Positive signals indicate the presence of stable anti-myc-TF-myc-ASIP complexes in the wells. As expected, only mixtures containing anti-myc-Tf and myc-ASIP exhibited signals in a dose-dependent manner with increasing myc-ASIP concentration, demonstrating the formation of a stable complex between anti-myc-Tf and myc-ASIP (FIG. 7d).

Delivery of Therapeutics by Intranasal Route to the Brain

Intranasal administration of therapeutics have been shown to effectively circumvent the BBB and deliver peptides and proteins successfully to various parts of the brain in non-human primates including the basal ganglia and substantia nigra (Thorne, 2008). For example, a patient with synuclein disease is laid in a supine position with the head titled back 45°, can be administered a bolus intranasal volume of 0.150 to 0.300 ml of ASIP or myc-ASIP dissolved in a solution per naris over approximately 1-2 min using a flexible plastic tubing. The patient remains in the same position for another 30 minutes to permit adequate absorption and delivery to the brain.

Modulating Expression Levels of MSH with Gene Silencing Technology

Antisense, Ribozyme, Triplex Techniques

The present invention provides a therapy for early intervention for synucleinopathy including PD, MSA and DLB by modifying expression levels of MSH. For example, in one non-limiting embodiment, treatment of synucleinopathy comprise of attenuating MSH expression levels with transcript targeting approaches.

One aspect of the invention relates to the use of purified antisense nucleic acids to inhibit expression of MSH. Antisense nucleic acid molecules within the invention described herein are those that specifically hybridize (e.g., bind) under cellular conditions to cellular mRNA encoding MSH in a manner that inhibits translation of MSH. The binding may be by conventional base pair complementarity. The nucleic acid molecules for use in the invention may hybridize to or partially hybridize to the polynucleotides shown herein as Ref seq ID numbers [α-MSH: SEQ ID NO: 11, β-MSH: SEQ ID NO: 14], which encode the peptides having the amino acid sequences of Ref seq ID numbers [α-MSH: SEQ ID NO: 12, β-MSH: SEQ ID NO: 15]. The invention also includes nucleic acids that may hybridize to or partially hybridize to portions of mRNA upstream to, overlapping with, or downstream of the nucleic acid sequence encoding the peptides having the amino acid sequences of Ref Seq ID numbers [α-MSH: SEQ ID NO: 12, β-MSH: SEQ ID NO: 15]. Nucleic acid sequences upstream and downstream of, and encoding the peptides shown herein as Ref Seq ID numbers [α-MSH: SEQ ID NO: 12, β-MSH: SEQ ID NO: 15] are shown shown herein as Ref seq ID numbers [α-MSH: SEQ ID NO: 10, β-MSH: SEQ ID NO: 13]. In some aspects of the invention, these polynucleotide sequences may be utilized for transcript targeting for inhibition of MSH expression.

Antisense constructs can be delivered, for example, as an expression plasmid which, when transcribed in the cell, produces RNA which is complementary to at least a unique portion of the cellular mRNA which encodes MSH. Alternatively, the antisense construct can take the form of an oligonucleotide probe generated ex vivo which, when introduced into MSH expressing cell, causes inhibition of MSH expression by hybridizing with an mRNA coding MSH. Such oligonucleotide probes are preferably modified oligonucleotides that are resistant to endogenous nucleases, e.g., exonucleases and/or endonucleases, and are therefore stable in vivo. Exemplary nucleic acid molecules for use as antisense oligonucleotides are phosphoramidate, phosphothioate and methylphosphonate analogs of DNA (see, e.g., U.S. Pat. Nos. 5,176,996; 5,264,564; and 5,256,775). Additionally, general approaches to constructing oligomers useful in antisense therapy have been reviewed, for example, by Van der Krol et al. (1988) Biotechniques 6:958-976; and Stein et al. (1988) Cancer Res 48:2659-2668. With respect to antisense DNA, oligodeoxyribonucleotides derived from the translation initiation site, e.g., between the −10 and +10 regions of MSH encoding nucleotide sequence, are preferred.

Antisense approaches involve the design of oligonucleotides that are complementary to MSH mRNA. The antisense oligonucleotides will bind to MSH transcripts and prevent translation. Absolute complementarity, although preferred, is not required. The ability to hybridize will depend on both the degree of complementarity and the length of the antisense nucleic acid. Generally, the longer the hybridizing nucleic acid, the more base mismatches with an RNA it may contain and still form a stable duplex or triplex. One skilled in the art can ascertain a tolerable degree of mismatch by use of standard procedures to determine the melting point of the hybridized complex. Oligonucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have been shown to be effective at inhibiting translation of mRNAs as well. (Wagner, R. (1994) Nature 372:333). Therefore, oligonucleotides complementary to either the 5′ or 3′ untranslated, non-coding regions of MSH gene could be used in an antisense approach to inhibit translation of endogenous MSH mRNA. Oligonucleotides complementary to the 5′ untranslated region of the mRNA should preferably include the complement of the AUG start codon. Although antisense oligonucleotides complementary to mRNA coding regions are generally less efficient inhibitors of translation, these could still be used in the invention. Generally, in order to be effective, the antisense oligonucleotide should be 18 or more nucleotides in length, but may be shorter depending on the conditions.

Specific antisense oligonucleotides can be tested for effectiveness using in vitro studies to assess the ability of the antisense oligonucleotide to inhibit gene expression. Preferably such studies (1) utilize controls (e.g., a non-antisense oligonucleotide of the same size as the antisense oligonucleotide) to distinguish between antisense gene inhibition and nonspecific biological effects of oligonucleotides, and (2) compare levels of the target protein with that of an internal control protein.

Oligonucleotides of the invention may be synthesized by standard methods known in the art, e.g., by use of an automated DNA synthesizer, as described above. Phosphorothioate oligonucleotides may be synthesized by the method of Stein et al. (1988) Nucl. Acids Res. 16:3209). Methylphosphonate oligonucleotides can be prepared by use of controlled pore glass polymer supports (e.g., as described in Sarin et al. (1988) Proc. Natl. Acad. Sci. U.S.A. 85:7448-7451).

Ribozymes

Ribozyme molecules designed to catalytically cleave MSH mRNA transcripts can also be used to prevent translation of MSH mRNAs and expression of MSH (see, e.g., Wright and Kearney, Cancer Invest. 19:495, 2001; Lewin and Hauswirth, Trends Mol. Med. 7:221, 2001; Sarver et al. (1990) Science 247:1222-1225 and U.S. Pat. No. 5,093,246). As one example, hammerhead ribozymes that cleave mRNAs at locations dictated by flanking regions that form complementary base pairs with the target mRNA might be used so long as the target mRNA has the following common sequence: 5′-UG-3′. See, e.g., Haseloff and Gerlach (1988) Nature 334:585-591. To increase efficiency and minimize the intracellular accumulation of non-functional mRNA transcripts, a ribozyme should be engineered so that the cleavage recognition site is located near the 5′ end of the target MSH mRNA. Ribozymes within the invention can be delivered to a cell using a vector as described below.

The antisense RNA and DNA, ribozyme, and triple helix molecules of the invention may be prepared by any method known in the art for the synthesis of DNA and RNA molecules. These include techniques for chemically synthesizing oligodeoxyribonucleotides and oligoribonucleotides well known in the art such as for example solid phase phosphoramide chemical synthesis. RNA molecules may be generated by in vitro and in vivo transcription of DNA sequences encoding the antisense RNA molecule. Such DNA sequences may be incorporated into a wide variety of vectors which incorporate suitable RNA polymerase promoters. Alternatively, antisense cDNA constructs that synthesize antisense RNA constitutively or inducibly, depending on the promoter used, can be introduced stably into cell lines.

RNA Interference (RNAi)

The use of short-interfering RNA (siRNA) is a technique known in the art for inhibiting expression of a target gene by introducing exogenous RNA into a living cell (Elbashir et al. 2001. Nature. 411:494-498). siRNAs suppress gene expression through a highly regulated enzyme-mediated process called RNA interference (RNAi). RNAi involves multiple RNA-protein interactions characterized by four major steps: assembly of siRNA with the RNA-induced silencing complex (RISC), activation of the RISC, target recognition and target cleavage. Therefore, identifying siRNA-specific features likely to contribute to efficient processing at each step is beneficial efficient RNAi. Reynolds et al. provide methods for identifying such features. A. Reynolds et al., “Rational siRNA design for RNA interference”, Nature Biotechnology 22(3), March 2004. In that study, eight characteristics associated with siRNA functionality were identified: low G/C content, a bias towards low internal stability at the sense strand 3′-terminus, lack of inverted repeats, and sense strand base preferences (positions 3, 10, 13 and 19). Further analyses revealed that application of an algorithm incorporating all eight criteria significantly improves potent siRNA selection. siRNA sequences that contain internal repeats or palindromes may form internal fold-back structures. These hairpin-like structures may exist in equilibrium with the duplex form, reducing the effective concentration and silencing potential of the siRNA. The relative stability and propensity to form internal hairpins can be estimated by the predicted melting temperatures (T_{M}). Sequences with high T_{M} values would favor internal hairpin structures.

siRNA can be used either ex vivo or in vivo, making it useful in both research and therapeutic settings. Unlike in other antisense technologies, the RNA used in the siRNA technique has a region with double-stranded structure that is made identical to a portion of the target gene, thus making inhibition sequence-specific. Double-stranded RNA-mediated inhibition has advantages both in the stability of the material to be delivered and the concentration required for effective inhibition.

The RNA used in this technique can comprise one or more strands of polymerized ribonucleotides, and modification can be made to the sugar-phosphate backbone as disclosed above. The double-stranded structure is often formed using either a single self-complementary RNA strand (hairpin) or two complementary RNA strands. RNA containing a nucleotide sequences identical to a portion of the target gene is preferred for inhibition, although sequences with insertions, deletions, and single point mutations relative to the target sequence can also be used for inhibition. Sequence identity may be optimized using alignment algorithms known in the art and through calculating the percent difference between the nucleotide sequences. The duplex region of the RNA could also be described in functional terms as a nucleotide sequence that is capable of hybridizing with a portion of the target gene transcript.

siRNA can often be a more effective therapeutic tool than other types of gene suppression due to siRNA's potent gene inhibition and ability to target receptors with a specificity can reach down to the level of single-nucleotide polymorphisms. Such specificity generally results in fewer side effects than is seen in conventional therapies, because other genes are not affected by application of a sufficiently sequence-specific siRNA.

There are multiple ways to deliver siRNA to the appropriate target. Standard transfection techniques may be used for in vitro systems, in which siRNA duplexes are incubated with cells of interest and then processed using standard commercially available kits. Electroporation techniques of transfection may also be appropriate. Cells can be soaked in a solution of the siRNA, allowing the natural uptake processes of the cells or organism to introduce the siRNA into the system. Viral constructs packaged into a viral particle would both introduce the siRNA into the cell line or organism and also initiate transcription through the expression construct. Other methods known in the art for introducing nucleic acids to cells may also be used, including lipid-mediated carrier transport, chemical-mediated transport, such as calcium phosphate, and the like.

Delivery vehicles for therapeutic uses include intravenous systemic delivery by transferrin-appended nucleic acid delivery vehicles or TfRMab decorated with liposomes containing RNA. Inhibition of gene expression can be confirmed by using biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell sorting (FACS). For RNA-mediated inhibition in a cell line or whole organism, gene expression may be assayed using a reporter or drug resistance gene whose protein product can be easily detected and quantified. Such reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentamycin, hygromycin, kanamycin, lincomycin, methotrexate, phosphinothricin, puromycin, and tetracyclin. These techniques are well known and easily practiced by those skilled in the art. For in vivo use in mammalian organisms, reduction or elimination of symptoms of illness will confirm inhibition of the target gene's expression.

CRISPR

In some aspects MSH gene expression can be silenced or decreased using the CRISPR-Cas13 system. Cas13a class enzymes can be used to attenuate gene expression at the RNA level as an approach for transcript targeting. A guide RNA complementary to the sequence of a section of the RNA coding for MSH may be designed to escort Cas13 to the portion of the nucleotide. Cas13 then cuts the targeted RNA, allowing knockdown of MSH, resulting in lower levels of MSH expression.

### EXAMPLES

Aspects of the present teachings may be further understood in light of the following examples, which should not be construed as limiting the scope of the present teachings in any way.

### Example 1—Animal Disease Model for Synucleinopathy

Three mice were given the inducer, α-MSH, through an intranasal route and one mouse was not subjected to α-MSH. The gait of all four mice was monitored every two weeks after subjection to one month of α-MSH treatment. No apparent leg impairment was observed in any mice two weeks after the end of treatment. Four, six, and eight weeks after administration of α-MSH the three mice given the α-MSH displayed progressive leg impairment. The impairment and weakening of the hind legs of the α-MSH treated mice resulted in loss of symmetry in gait, a shift in the center of gravity inward (FIG. 5b. week 4, circle), and compensation by the non-impaired leg and its movement inward (FIG. 5b, week 4, circle). The single control mouse not subjected to α-MSH maintained normal gait. Gait impairment observed in the three mice given the α-MSH inducer is characteristic of motor symptoms associated with PD and MSA symptoms. Gait impairment became more severe with time, indicating a progressively worsening disease state consistent with synucleinopathy. Moreover, synuclein pathology was observed in the substantia nigra (FIG. 6) of the mouse described in FIG. 5b. Thus, this animal disease model was induced by exposure of the brain to α-MSH.

### Example 2—Cellular Model of Parkinson's Disease/Multiple System Atrophy

A human pigmented cell line MNT-1 was utilized as a model for pigmented dopaminergic neurons to assess synuclein pathology following exposure to the inducer, α-MSH. After subjecting the cells to α-MSH exposure, accumulation and aggregation of α-synuclein (FIG. 2B) and lack of melanin were observed in the cells (FIG. 1B). Additionally, cell autophagy induced by rapamycin or starvation was disabled (FIGS. 1A and 1B). Impaired autophagy results in a failure to slow down glucose consumption in response to depleted nutrient conditions (FIG. 4A). With time, vulnerable cells begin to die and undergo apoptotic cell death as measured by caspase activity (FIG. 4B). α-Synuclein accumulation and aggregation, loss of melanin and cell death by apoptosis are cardinal features observed in synuclein diseases such as PD and MSA.

One skilled in the art can recognize the cellular events following exposure to α-MSH as cardinal features in PD, MSA and DLB cellular pathology, and that it was α-MSH that was responsible for inducing the disease, since cells not exposed to α-MSH remained normal and healthy (FIG. 2A). Indeed, the loss of neuromelanin in dopaminergic neurons in the substantia nigra, accumulation and aggregation of α-synuclein in Lewy body formations, and cell death by apoptosis are classic observances in PD and MSA (Xu, 2015; Yasuda, 2013). Exposure to MSH initiates the pathway to synucleinopathy and this can be exploited to manufacture animal disease models.

### Example 3—Clinical Observations Implicate MSH in Synucleinopathy

Microglial cells are activated in the brains of patients with synuclein disease (Ishizawa, 2004; Croisier 2005, Olanow 2019) and α-MSH, an anti-inflammatory mediator, is abundantly produced and released by activated microglia (Delgado, 1998). Indeed, in patients with PD or MSA, an elevated level of α-MSH is found in cerebrospinal fluid. A 2-fold increase of α-MSH is seen compared to healthy control patients, as measured by immunoreactivity (Rainero, 1988; Konagaya 1991). Treatment with the tripeptide Pro-Leu-Gly-NH₂ (PLG, Melanocyte Inhibiting Factor 1, or MIF-1), a known suppressor of hypothalamic release of α-MSH, results in improvement in disease symptoms (Barbeau, 1976). Similarly, treatment with melatonin, which inhibits α-MSH activity (Valverde, 1995), resulted in improved PD symptoms (as described by Dr. George C. Cotzias himself in a filming interview (Fahn, 2015) and stalled locomotor deficits in Parkinson's mouse models (Patki, 2011). Conversely, patients administered β-MSH (similar to α-MSH) showed worsening symptoms (Cotzias, 1967).

### Example 4—ASIP as a Therapeutic for Synucleinopathy

Inhibiting MSH activity by antagonizing its engagement to receptors, including, but not limited to, MC1R, is a therapeutic method for relieving and reversing synuclein disease progression. ASIP, an endogenous antagonist for MSH binding to receptors, including MC1R, is shown in this invention to halt α-synuclein accumulation and aggregation induced by α-MSH (FIG. 2C). More importantly ASIP reverses the effects of α-MSH by restoring melanin production (FIG. 3). ASIP also protects against cell death, blocking the deleterious effects of α-MSH (FIG. 4C). Thus, as a therapeutic ASIP promotes an environment ideal for recovery and cell restoration strategies.

### REFERENCES CITED

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

Specifically intended to be within the scope of the present invention, and incorporated herein by reference in its entirety, is the following publication:
1. Lynch-Day M. A., Mao, K., Wang, K., Zhao, M., Klionsky, D. J. (2012) Cold Spring Harb Perspect Med 2, a009357.
2. Dickson, D. W. (2012) Cold Spring Harb Perspect Med 2, a009258.
3. Flabeau, O., Meissner, W. G., Tison, F. (2010) Ther Adv Neurol Disord 3, 249-263.
4. Klein, C., Westenberger, A. (2012) Cold Spring Harb Perspect Med 2, a008888.
5. Sharma, S., Moon, C. S., Khogali, A., Haidous, A., Chabenne, A., Ojo, C., Jelebinkov, M., Kurdi, Y., Ebadi M., (2013) Neurochem Int 63, 201-229.
6. Miller, D. B., O'Callaghan, J. P. (2015) Metabolism 64, S40-S46.
7. Brooks, D. (2000) J Neurol Neurosurg Psychiatry 68, 685-689.
8. Blesa, J., Przedborski. S. (2014) Front Neuroanat 8, DOI:10.3389/fnana.2014.00155.
9. Cotzias, G. C., Van Woert, M. H., Schiffer, L. M. (1967) N Engl J Med 267, 374-379.
10. Fahn, S. (2015) Movement Disord 30, 4-18.
11. Sato, S., Uchihara, T., Fukuda, T., Noda, S., Kondo, H., Saiki, S., Komatsu, M., Uchiyama, Y., Tanaka, K., Hattori, N. (2018) Sci Rep DOI:10.1038/s41598-018-21325-w.
12. Anglade, P., Vyas, S., Javoy-Agid, F., Herrero, M. T., Michel, P., Marquez, J., Mouatt-Prigent, A., Ruberg, M., Hirsch, E. C, Agid, Y. (1997) Histol Histopathol 12(1), 25-31.
13. Chu, Y., Goldman, J., Kelly, L., He, Y., Waliczek, T., Kordower, J. H. (2014) Neurobiol Dis 69, 1-14.
14. Lill, C. M. (2016) Mol Cell Probes 30(6), 386-396.
15. Manzoni, C., Lewis, P. A. (2013) FASEB J 27(9), 3424-3429.
16. Beilina, A., Cookson, M. R. (2016) J Neurochem 139(1), 91-107.
17. Kalie, E., Razi M., Tooze S. A. (2013) PLoS One 8(9):e75313.
18. Zhang, C., Gruber, F., Ni, C., Mildner, M., Koenig, U., Karner, S., Barresi, C., Rossiter, H., Narzt, M., Nagelreiter, I. M., Larue, L., Tobin, D. J., Eckhart, L., Tschachler, E. (2015) J Invest Dermatol 135, 1348-1357.
19. Xu, S., Chan, P. (2015) Biomolecules 5, 1122-1142.
20. Yasuda, T., Nakata, Y., Mochizuki, H. (2013) Mol Neurobiol 47, 466-483.
21. Chen, X., Chen, H., Cai, W., Maguire, M., Ya, B., Zuo, F., Logan, R., Li, H., Robinson, K., Vanderburg. C. R., Yu, Y., Wang, Y., Fisher, D. E., Schwarzschild M. A. (2017) Ann Neurol 81(3), 395-406.
22. Wolf Horrell, E. M., Boulanger, M. C., D'Orazio, J. A. (2016) Front Genet 7, DOI:10.3389/fgene.2016.00095.
23. Chai, B., Neubig, R. R., Millhauser, G. L., Thompson, D. A., Jackson, P. J., Barsh, G. S., Dickinson, C. J., Li, J., Lai, Y., Gantz, I. (2003) Peptides 24, 603-609.
24. Jackson, P. J., Douglas, N. R., Chai, B., Binkley, J., Sidow, A., Barsh, G. S., Millhauser, G. L. (2006) Chem Biol 13, 1297-1305.
25. Kastin, A. J., Akerstrom, V., Heckler, L. (2000) Metabolism 49(11), 1444-1448.
26. Patel, M. P., Cribb Fabersunne, C. S., Yang, Y. K., Kaelin, C. B., Barsh, G. S., Millhauser G. L. (2010) J Mol Biol 404(1), 45-55.
27. Pardridge, W. M., (2015) Expert Opin Drug Deliv 12, 207-222.
28. Shin, S., Friden, P., Moran, M>, Olson, T., Kang, Y., Pardridge, W. M., Morrison, S. L. (1995) Proc Natl Acad Sci 92, 2820-2824.
29. Paterson, J., Webster, C. I., (2016) Drug Discov Today Technol 20, 49-52.
30. Schiweck, W., Buxbaum, B., Schatzlein, C., Neiss, H., Skerra A. (1997) FEBS Lett 414(1), 33-38.
31. Thorne, R. G., Hanson, L. R., Ross, T. M., Tung, D., Frey, W. H. 2nd. (2008) Neuroscience 152(3), 785-797.
32. Ishizawa, K., Komori, T., Sasaki, S., Arai, N., Mizutani, T., Hirose, T. (2004) J. Neuropathol Exp Neurol 65, 43-54.
33. Croisier, E., Moran, L. B., Dexter, D. T., Pearce R. K., Graeber, M. B. (2005). J Neuroinflammation 2, doi:10.1186/1742-2094-2-14.
34. Delgado, R., Carlin, A., Airaghi, L., Demitri, M. T., Meda, L., Galimberti, D., Baron, P., Lipton, J. M., Catania, A. (1998) J Leukoc Biol 740-5
35. Olanow, C. W., Savolainen, M., Chu, Y., Halliday, G. M., Kordower, J. H. (2019) Brain 142(6), 1690-1700.
36. Rainero, I., Kaye, J. A., May, C., Durso, R., Katz, D. I., Albert, M. L., Wolfe, N., Pinessi, L., Friedland, R. P., Rapoport, S. I. (1988) Arch Neurol 1224-1227.
37. Konagaya, Y., Konagaya, M., (1991) Rinsho Shinkeigaku 31(8), 821-825.
38. Barbeau, A., Kastin, A. J. (1976) Advances in Parkinsonism 483-487.
39. Valverde, P., Healy, E., Jackson, I., Rees, J. L., Thody, A. J. (1995) Nat Genet 11, 328-330.
40. Patki, G., Lau Y. (2011) Pharmacol Biochem Behav 99, 704-711.

### SEQ ID NO: LISTING

1. Agouti Signaling Protein (ASIP), mRNA [Homo Sapiens]: NCBI Reference Sequence: NM_001672.2 (SEQ ID No 1):

GCCTCCTGGGATGGATGTCACCCGCTTACTCCTGGCCACCCTGCTGGTCTT
CCTCTGCTTCTTCACTGCCAACAGCCACCTGCCACCTGAGGAGAAGCTCCG
AGATGACAGGAGCCTGAGAAGCAACTCCTCTGTGAACCTACTGGATGTCCC
TTCTGTCTCTATTGTGGCGCTGAACAAGAAATCCAAACAGATCGGCAGAAA
AGCAGCAGAAAAGAAAAGATCTTCTAAGAAGGAGGCTTCGATGAAGAAAGT
GGTGCGGCCCCGGACCCCCCTATCTGCGCCCTGCGTGGCCACCCGCAACAG
CTGCAAGCCGCCGGCACCCGCCTGCTGCGACCCGTGCGCCTCCTGCCAGTG
CCGCTTCTTCCGCAGCGCCTGCTCCTGCCGCGTGCTCAGCCTCAACTGCTG
AGCGCCCCCACTCCCGGCCGCGAGCAGGCAGGGCTTCGGGGACGCGGGGCG
CTTCTCGGGCGGGTGATCCCTAACAGGGCGGCTTCCCAGGGCTGCAGGCGG
GCGGAGGTTCCAGGAGATGGGACTTCAGGGAGACCTGGCTTGGGCTAAAAT
CGAAATACAATATATATAGGCTG
https://www.ncbi.nlm.nih.gov/nuccore/21327679

2. Agouti Signaling Protein (ASIP), mRNA [Homo Sapiens]: (SEQ ID NO: 2)

TGGATGTCACCCGCTTACTCCTGGCCACCCTGCTGGTCTTCCTCTGCTTCT
TCACTGCCAACAGCCACCTGCCACCTGAGGAGAAGCTCCGAGATGACAGGA
GCCTGAGAAGCAACTCCTCTGTGAACCTACTGGATGTCCCTTCTGTCTCTA
TTGTGGCGCTGAACAAGAAATCCAAACAGATCGGCAGAAAAGCAGCAGAAA
AGAAAAGATCTTCTAAGAAGGAGGCTTCGATGAAGAAAGTGGTGCGGCCCC
GGACCCCCCTATCTGCGCCCTGCGTGGCCACCCGCAACAGCTGCAAGCCGC
CGGCACCCGCCTGCTGCGACCCGTGCGCCTCCTGCCAGTGCCGCTTCTTCC
GCAGCGCCTGCTCCTGCCGCGTGCTCAGCCTCAACTGCT
https://www.ncbi.nlm.nih.gov/nuccore/21327679

3. Agouti-Signaling Protein Precursor (ASIP) [Homo Sapiens]: NCBI Reference Sequence: NP_001663.2 (SEQ ID NO: 3)

MDVTRLLLATLLVFLCFFTANSHLPPEEKLRDDRSLRSNSSVNLLDVPSVS
IVALNKKSKQIGRKAAEKKRSSKKEASMKKVVRPRTPLSAPCVATRNSCKP
PAPACCDPCASCQCRFFRSACSCRVLSLNC
https://www.ncbi.nlm.nih.gov/protein/NP_001663.2

4. Agouti Related Neuropeptide (AGRP), mRNA [Homo Sapiens]: NCBI Reference Sequence: NM_001138.1 (SEQ ID No: 4)

AGCTCCTAGGTCCCTGTCCTGTGGAAATTTGTGGACCCTGGGCACCCTCTC
TTGCTCCCAAATTTTAATCGGCTCCTGGAAACCTCACCCCAAATTGGAGAT
AGGCACTCCTCTTGTAGAACAAAAGGCTCAGGTTCAGGGAGTGAGGGCCTG
AACTGTGCCCCCACCCTCCAGGAAGGGTCCTTCACGGCCTGGCTGCAGGGA
TCAGTCACGTGTGGCCCTTCATTAGGCCCTGCCATATAAGCCAAGGGCACG
GGGTGGCCGGGAACTCTCTAGGCAAGAATCCCGGAGGCAGAGGCCATGCTG
ACCGCAGCGGTGCTGAGCTGTGCCCTGCTGCTGGCACTGCCTGCCACGCGA
GGAGCCCAGATGGGCTTGGCCCCCATGGAGGGCATCAGAAGGCCTGACCAG
GCCCTGCTCCCAGAGCTCCCAGGCCTGGGCCTGCGGGCCCCACTGAAGAAG
ACAACTGCAGAACAGGCAGAAGAGGATCTGTTGCAGGAGGCTCAGGCCTTG
GCAGAGGTACTAGACCTGCAGGACCGCGAGCCCCGCTCCTCACGTCGCTGC
GTAAGGCTGCATGAGTCCTGCCTGGGACAGCAGGTGCCTTGCTGTGACCCA
TGTGCCACGTGCTACTGCCGCTTCTTCAATGCCTTCTGCTACTGCCGCAAG
CTGGGTACTGCCATGAATCCCTGCAGCCGCACCTAGCTGGCCAACGTCAGG
GTCGGGGCTAGGGTAGGGGCAAGGAAGGACCAACAAAAAAAAAAAAAAAAA
AA
https://www.ncbi.nlm.nih.gov/nuccore/4501994

5. Agouti Related Neuropeptide (AgRP), mRNA [Homo Sapiens]: (SEQ ID NO: 5)

TGCTGACCGCAGCGGTGCTGAGCTGTGCCCTGCTGCTGGCACTGCCTGCCA
CGCGAGGAGCCCAGATGGGCTTGGCCCCCATGGAGGGCATCAGAAGGCCTG
ACCAGGCCCTGCTCCCAGAGCTCCCAGGCCTGGGCCTGCGGGCCCCACTGA
AGAAGACAACTGCAGAACAGGCAGAAGAGGATCTGTTGCAGGAGGCTCAGG
CCTTGGCAGAGGTACTAGACCTGCAGGACCGCGAGCCCCGCTCCTCACGTC
GCTGCGTAAGGCTGCATGAGTCCTGCCTGGGACAGCAGGTGCCTTGCTGTG
ACCCATGTGCCACGTGCTACTGCCGCTTCTTCAATGCCTTCTGCTACTGCC
GCAAGCTGGGTACTGCCATGAATCCCTGCAGCCGCACCT
https://www.ncbi.nlm.nih.gov/nuccore/4501994

6. Agouti-Related Protein Precursor (AgRP) [Homo Sapiens]: NCBI Reference Sequence: NP_001129.1 (SEQ ID NO: 6)

MLTAAVLSCALLLALPATRGAQMGLAPMEGIRRPDQALLPELPGLGLRAPL
KKTTAEQAEEDLLQEAQALAEVLDLQDREPRSSRRCVRLHESCLGQQVPCC
DPCATCYCRFFNAFCYCRKLGTAMNPCSRT
https://www.ncbi.nlm.nih.gov/protein/NP_001129.1

7. Defensin beta 103A (DEFB103A) (HBD3), mRNA [Homo Sapiens]: NCBI Reference Sequence: NM_001081551.3 (SEQ ID No: 7)

CAAATCCATAGGGAGCTCTGCCTTACCATTGGGTTCCTAATTAACTGAGTG
AGTGGGTGTGTTCTGCATGGTGAGAGGCATTGGAATGATGCATCAGAAAAC
ATGTCATAATGTCATCACTGTAATATGACAAGAATTGCAGCTGTGGCTGGA
ACCTTTATAAAGTGACCAAGCACACCTTTTCATCCAGTCTCAGCGTGGGGT
GAAGCCTAGCAGCTATGAGGATCCATTATCTTCTGTTTGCTTTGCTCTTCC
TGTTTTTGGTGCCTGTTCCAGGTCATGGAGGAATCATAAACACATTACAGA
AATATTATTGCAGAGTCAGAGGCGGCCGGTGTGCTGTGCTCAGCTGCCTTC
CAAAGGAGGAACAGATCGGCAAGTGCTCGACGCGTGGCCGAAAATGCTGCC
GAAGAAAGAAATAAAAACCCTGAAACATGACGAGAGTGTTGTAAAGTGTGG
AAATGCCTTCTTAAAGTTTATAAAAGTAAAATCAAATTACATTTTTTTTTC
AAAAAAAA
https://www.ncbi.nlm.nih.gov/nuccore/611434968

8. Defensin beta 103A (DEFB103A) (HBD3), mRNA [Homo Sapiens]: (SEQ ID No: 8)

TGAGGATCCATTATCTTCTGTTTGCTTTGCTCTTCCTGTTTTTGGTGCCTG
TTCCAGGTCATGGAGGAATCATAAACACATTACAGAAATATTATTGCAGAG
TCAGAGGCGGCCGGTGTGCTGTGCTCAGCTGCCTTCCAAAGGAGGAACAGA
TCGGCAAGTGCTCGACGCGTGGCCGAAAATGCTGCCGAAGAAAGAAAT
https://www.ncbi.nlm.nih.gov/nuccore/611434968

9. Beta-defensin 103 (HBD3) precursor [Homo Sapiens]: NCBI Reference Sequence: NP_001075020.1 (SEQ ID NO: 9)

MRIHYLLFALLFLFLVPVPGHGGIINTLQKYYCRVRGGRCAVLSCLPKEEQ
IGKCSTRGRKCCRRKK
https://www.ncbi.nlm.nih.gov/protein/NP_001075020.1

10. Proopiomelanocortin (POMC), transcript variant 2 (α-MSH), mRNA [Homo Sapiens]: NCBI Reference Sequence: NM_000939.3 (SEQ ID No: 10)

GTTCTAAGCGGAGACCCAACGCCATCCATAATTAAGTTCTTCCTGAGGGCG
AGCGGCCAGGTGCGCCTTCGGCAGGACAGTGCTAATTCCAGCCCCTTTCCA
GCGCGTCTCCCCGCGCTCGTCCCCCGTCTGGAAGCCCCCCTCCCACGCCCC
GCGGCCCCCCTTCCCCTGGCCCGGGGAGCTGCTCCTTGTGCTGCCGGGAAG
GTCAAAGTCCCGCGCCCACCAGGAGAGCTCGGCAAGTATATAAGGACAGAG
GAGCGCGGGACCAAGCGGCGGCGAAGGAGGGGAAGAAGAGCCGCGACCGAG
AGAGGCCGCCGAGCGTCCCCGCCCTCAGAGAGCAGCCTCCCGAGACAGAGC
CTCAGCCTGCCTGGAAGATGCCGAGATCGTGCTGCAGCCGCTCGGGGGCCC
TGTTGCTGGCCTTGCTGCTTCAGGCCTCCATGGAAGTGCGTGGCTGGTGCC
TGGAGAGCAGCCAGTGTCAGGACCTCACCACGGAAAGCAACCTGCTGGAGT
GCATCCGGGCCTGCAAGCCCGACCTCTCGGCCGAGACTCCCATGTTCCCGG
GAAATGGCGACGAGCAGCCTCTGACCGAGAACCCCCGGAAGTACGTCATGG
GCCACTTCCGCTGGGACCGATTCGGCCGCCGCAACAGCAGCAGCAGCGGCA
GCAGCGGCGCAGGGCAGAAGCGCGAGGACGTCTCAGCGGGCGAAGACTGCG
GCCCGCTGCCTGAGGGCGGCCCCGAGCCCCGCAGCGATGGTGCCAAGCCGG
GCCCGCGCGAGGGCAAGCGCTCCTACTCCATGGAGCACTTCCGCTGGGGCA
AGCCGGTGGGCAAGAAGCGGCGCCCAGTGAAGGTGTACCCTAACGGCGCCG
AGGACGAGTCGGCCGAGGCCTTCCCCCTGGAGTTCAAGAGGGAGCTGACTG
GCCAGCGACTCCGGGAGGGAGATGGCCCCGACGGCCCTGCCGATGACGGCG
CAGGGGCCCAGGCCGACCTGGAGCACAGCCTGCTGGTGGCGGCCGAGAAGA
AGGACGAGGGCCCCTACAGGATGGAGCACTTCCGCTGGGGCAGCCCGCCCA
AGGACAAGCGCTACGGCGGTTTCATGACCTCCGAGAAGAGCCAGACGCCCC
TGGTGACGCTGTTCAAAAACGCCATCATCAAGAACGCCTACAAGAAGGGCG
AGTGAGGGCACAGCGGGGCCCCAGGGCTACCCTCCCCCAGGAGGTCGACCC
CAAAGCCCCTTGCTCTCCCCTGCCCTGCTGCCGCCTCCCAGCCTGGGGGGT
CGTGGCAGATAATCAGCCTCTTAAAGCTGCCTGTAGTTAGGAAATAAAACC
TTTCAAATTTCACATCCACCTCTGACTTTGAATGTAAACTGTGTGAATAAA
GTAAAAATACGTAGCCGTCAAATAACAGC
https://www.ncbi.nlm.nih.gov/nuccore/NM_000939

11. Proopiomelanocortin (POMC), transcript variant 2 (α-MSH), mRNA [Homo Sapiens]: (SEQ ID NO: 11)

TCCTACTCCATGGAGCACTTCCGCTGGGGCAAGCCGGTGG
https://www.ncbi.nlm.nih.gov/nuccore/NM_000939

12. α-Melanocyte Stimulating Hormone (α-MSH): Accession No PRO_0000024970 (SEQ ID NO: 12)

SYSMEHFRWGKPV
https://www.genome.jp/dbget-bin/
www_bget?uniprot:COLI_HUMAN

13. Proopiomelanocortin (POMC) (β-MSH), mRNA [Homo Sapiens]: NCBI Reference Sequence: NM_000939.3 (SEQ ID No: 13)

GTTCTAAGCGGAGACCCAACGCCATCCATAATTAAGTTCTTCCTGAGGGCG
AGCGGCCAGGTGCGCCTTCGGCAGGACAGTGCTAATTCCAGCCCCTTTCCA
GCGCGTCTCCCCGCGCTCGTCCCCCGTCTGGAAGCCCCCCTCCCACGCCCC
GCGGCCCCCCTTCCCCTGGCCCGGGGAGCTGCTCCTTGTGCTGCCGGGAAG
GTCAAAGTCCCGCGCCCACCAGGAGAGCTCGGCAAGTATATAAGGACAGAG
GAGCGCGGGACCAAGCGGCGGCGAAGGAGGGGAAGAAGAGCCGCGACCGAG
AGAGGCCGCCGAGCGTCCCCGCCCTCAGAGAGCAGCCTCCCGAGACAGAGC
CTCAGCCTGCCTGGAAGATGCCGAGATCGTGCTGCAGCCGCTCGGGGGCCC
TGTTGCTGGCCTTGCTGCTTCAGGCCTCCATGGAAGTGCGTGGCTGGTGCC
TGGAGAGCAGCCAGTGTCAGGACCTCACCACGGAAAGCAACCTGCTGGAGT
GCATCCGGGCCTGCAAGCCCGACCTCTCGGCCGAGACTCCCATGTTCCCGG
GAAATGGCGACGAGCAGCCTCTGACCGAGAACCCCCGGAAGTACGTCATGG
GCCACTTCCGCTGGGACCGATTCGGCCGCCGCAACAGCAGCAGCAGCGGCA
GCAGCGGCGCAGGGCAGAAGCGCGAGGACGTCTCAGCGGGCGAAGACTGCG
GCCCGCTGCCTGAGGGCGGCCCCGAGCCCCGCAGCGATGGTGCCAAGCCGG
GCCCGCGCGAGGGCAAGCGCTCCTACTCCATGGAGCACTTCCGCTGGGGCA
AGCCGGTGGGCAAGAAGCGGCGCCCAGTGAAGGTGTACCCTAACGGCGCCG
AGGACGAGTCGGCCGAGGCCTTCCCCCTGGAGTTCAAGAGGGAGCTGACTG
GCCAGCGACTCCGGGAGGGAGATGGCCCCGACGGCCCTGCCGATGACGGCG
CAGGGGCCCAGGCCGACCTGGAGCACAGCCTGCTGGTGGCGGCCGAGAAGA
AGGACGAGGGCCCCTACAGGATGGAGCACTTCCGCTGGGGCAGCCCGCCCA
AGGACAAGCGCTACGGCGGTTTCATGACCTCCGAGAAGAGCCAGACGCCCC
TGGTGACGCTGTTCAAAAACGCCATCATCAAGAACGCCTACAAGAAGGGCG
AGTGAGGGCACAGCGGGGCCCCAGGGCTACCCTCCCCCAGGAGGTCGACCC
CAAAGCCCCTTGCTCTCCCCTGCCCTGCTGCCGCCTCCCAGCCTGGGGGGT
CGTGGCAGATAATCAGCCTCTTAAAGCTGCCTGTAGTTAGGAAATAAAACC
TTTCAAATTTCACATCCACCTCTGACTTTGAATGTAAACTGTGTGAATAAA
GTAAAAATACGTAGCCGTCAAATAACAGC
https://www.ncbi.nlm.nih.gov/nuccore/NM_000939

14. Proopiomelanocortin (POMC) (β-MSH), mRNA [Homo Sapiens]: (SEQ ID NO: 14)

ACGAGGGCCCCTACAGGATGGAGCACTTCCGCTGGGGCAGCCCGCCCAAGG
ACA
https://www.ncbi.nlm.nih.gov/nuccore/NM_000939

15. β-Melanocyte Stimulating Hormone (β-MSH) Accession No. PRO_0000024974 (SEQ ID NO: 15)

DEGPYRMEHFRWGSPPKD
https://www.genome.jp/dbget-bin/
www_bget?uniprot:COLI_HUMAN

1-5. (canceled)

6. A method of identifying a molecule as a candidate therapeutic for a synucleinopathy, wherein the synucleinopathy is selected from the group consisting of Parkinson's Disease, Multiple System Atrophy and Dementia with Lewy bodies, the method comprising the steps of:
a) providing an animal having a synucleinopathy-related condition induced by administering to the animal a Melanocyte Stimulating Hormone (MSH), wherein the MSH is selected from the group consisting of α-MSH and β-MSH, and the synucleinopathy-related condition is one or more conditions selected from the group consisting of aggregation of α-synuclein, loss of autophagy, loss of neuronal function and pigmentation, loss of cell viability, and loss of neurotransmitters in a brain of the animal;
b) measuring in the brain of the animal at least one of the parameters selected from the group consisting of aggregation of α-synuclein, loss of autophagy, loss of neuronal function and pigmentation, loss of cell viability and loss of neurotransmitters;
c) measuring loss of a motor control parameter or a motor deficit parameter for the animal;
d) administering a molecule to the animal having the synucleinopathy-related condition;
e) repeating the measuring steps (b) and (c) to the animal after administration of the molecule;
and
f) identifying the molecule as a candidate therapeutic for the synucleinopathy when at least one of the parameters measured at the steps (b) and (c) is decreased during measuring at the step (e).

7. The method of claim 6, wherein the molecule is identified as a candidate therapeutic for the synucleinopathy when at least one of the parameters measured at the step (b) and at least one of the parameters measured at the step (c) are decreased during measuring at the step (e).

8. A method of treating, preventing, inhibiting or reversing a synucleinopathy in an animal, wherein the synucleinopathy is selected from the group consisting of Parkinson's Disease, Multiple System Atrophy and Dementia with Lewy bodies, the method comprising administering to the animal a compound that attenuates binding of a Melanocyte Stimulating Hormone (MSH) to its receptor, wherein the MSH is selected from the group consisting of α-MSH and β-MSH.

9. The method of claim 8, wherein upon administration, the compound antagonizes an activity of the MSH.

10. (canceled)

11. The method of claim 8, wherein the receptor is a Melanocortin receptor.

12-14. (canceled)

15. The method of claim 8, wherein the compound is a protein or a mutant, variant or fragment thereof, wherein the protein is selected from the group consisting of Agouti Signaling Protein (ASIP), Agouti Related protein (AgRP), and human β-defensin3 (HBD3).

16. The method of claim 8, wherein the compound is a protein or a mutant, variant or fragment thereof, wherein the protein is an antibody that binds to the MSH and a) inhibits an MSH activity and/or b) diminishes a level of the MSH.

17. The method of claim 8, wherein the compound is a small molecule that binds to the MSH and a) inhibits an MSH activity and/or b) diminishes a level of the MSH.

18-24. (canceled)

25. A method for treating, preventing, inhibiting or reversing synucleinopathy in an mammal, wherein the synucleinopathy is selected from the group consisting of Parkinson's Disease, Multiple System Atrophy and Dementia with Lewy bodies, the method comprising administering to the mammal a compound that binds to a RNA transcript molecule encoding a Melanocyte Stimulating Hormone (MSH) and attenuates an expression of MSH, wherein the MSH is selected from the group consisting of α-MSH and β-MSH.

26-28. (canceled)

29. The method according to claim 25 wherein the compound is selected from the group consisting of an antisense nucleic acid, a ribozyme, a triplex-forming oligonucleotide, a siRNA, a primer, a CRISPR molecule, and any combination thereof.

30. (canceled)

31. The method according to claim 25 wherein the compound comprises a nucleic acid that hybridizes with the RNA transcript molecule encoding MSH and attenuates the expression of MSH.

32-37. (canceled)

38. The method according to claim 25, wherein the compound has at least one modified internucleoside linkage, sugar moiety, or nucleobase.

39. The method according to claim 25, wherein the compound is an siRNA.

40. (canceled)

41. The method according to claim 39, wherein the siRNA comprises a RNA lacking stable internal repeats.

42. The method according to claim 39, wherein the siRNA comprises a hairpin RNA.

43. The method according to claim 25, wherein the transcript encoding MSH is altered by introducing into cells containing MSH nucleic acids an engineered non-naturally occurring Clustered Regularly Interspaced Short Palindromic Repeats (CRISPR)-CRISPR associated (Cas) (CRISPR-Cas) system.

44. (canceled)
